SEQUENCE LISTING (11 sequences)

SEQ ID NO: 1 - protein, 189 aa, Homo sapiens [sequence omitted]

SEQ ID NO: 2 - protein, 372 aa, Homo sapiens
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 3 - protein, 225 aa, Homo sapiens
MDLRAGDSWGMLACLCTVLWHLPAVPALNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 4 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 5 - protein, 204 aa, Homo sapiens
MYCLLATPLCLLSLLLPPLSPAAPISPSEPIGQAYSLALYMQKNTSALLQTYLQHQGSPFSDPGFSAPELQLSTLPSAAVSFKTWHAMEDAERLSRAQGAFLALTQHLQLVGDDQSYLNPGSPILLAQLGAARLRAQGLLGNMAAIMTALGLPIPPEEDTLGFVPFGASAFERKCRGYIVTREYGHWTDRAVRDLALLKAKYSA

SEQ ID NO: 6 - protein, 198 aa, Artificial Sequence
  synthetic sequence
LNRTGDPGPGPSIQKTYDLTRYLEHQLRSLAGTYLNYLGPPFNEPDFNPPRLGAETLPRATVDLEVWRSLNDKLRLTQNYEAYSHFLCYLRGLNRRAATAELRRSLAHFCTSLQGLLGSIAGVMAALGYPLPQPLPGTEPTWTPGPARSDFLQKMDDFWLLKELQTWLWRSAKDFNRLKKKMQPPAAAVTLHLGAHGF

SEQ ID NO: 7 - protein, 198 aa, Artificial Sequence
  synthetic sequence
LNRTGDPGPGPSIQKTYDLTRCLEHQLRSLAGTYLNYLGPPFNEPDFNPPRLGAETLPRATVDLEVWRSLNDKLRLTQNYEAYSHFLCYLRGLNRRAATAELRRSLAHFCTSLQGLLGSIAGVMAALGYPLPQPLPGTEPTWTPGPARSDALQAMDDFWLLKELQTWLLRSAKDFNRLKRKMQPPAAAVTLHLGAHGF

SEQ ID NO: 8 - protein, 372 aa, Homo sapiens; identical to SEQ ID NO: 2

SEQ ID NO: 9 - protein, 342 aa, Artificial Sequence
  synthetic sequence
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPQEPVLSCRSNTYPKGFYCSWHLPTPTYIPNTFNVTVLHGSKIMVCEKDPALKNRCHIRYMHLFSPIKHNVSISVSNALGHNATAITFDEFTIVKPDPPENVVARPVPSNPRRLEVTWQTPSTWPDPEFFPLKFFLRYRPLILDQWQHVELSDGTAHTIAAAYAGKEYIIQVAAKDNEFGTWSDWSVAAHATPWTEEPRHLTTEAQAAETTTSTTSSLAPPPTTKICDPGELGS

SEQ ID NO: 10 - protein, 578 aa, Artificial Sequence
  synthetic sequence
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPQEPVLSCRSNTYPKGFYCSWHLPTPTYIPNTFNVTVLHGSKIMVCEKDPALKNRCHIRYMHLFSPIKHNVSISVSNALGHNATAITFDEFTIVKPDPPENVVARPVPSNPRRLEVTWQTPSTWPDPEFFPLKFFLRYRPLILDQWQHVELSDGTAHTIAAAYAGKEYIIQVAAKDNEFGTWSDWSVAAHATPWTEEPRHLTTEAQAAETTTSTTSSLAPPPTTKICDPGELGSRRLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0331401 A1
(43) Pub. Date: Oct. 20, 2022

(54) METHODS OF TREATING KRAS MUTANT CANCERS

(71) Applicants: The Board of Trustees of the Leland Stanford Junior University, Stanford, CA (US); The Regents of the University of California, Oakland, CA (US)

(72) Inventors: Eric Alejandro Sweet-Cordero, San Francisco, CA (US); Jennifer R. Cochran, Stanford, CA (US); Jun Woo Kim, Mountain View, CA (US); Cesar P. Marquez, Stanford, CA (US)

(21) Appl. No.: 17/639,525

(22) PCT Filed: Sep. 10, 2020
(86) PCT No.: PCT/US2020/050206

§ 371 Date: Mar. 1, 2022

Related U.S. Application Data
(60) Provisional application No. 62/931,608, filed on Nov. 6, 2019.
(60) Provisional application No. 62/898,249, filed on Sep. 10, 2019.

(51) Int. Cl. A61K 38/17; A61P 35/00; A61P 11/00; A61K 38/19; A61K 47/60

(52) U.S. Cl. CPC A61K 38/1793; A61P 35/00; A61P 11/00; A61K 38/19; A61K 47/60

(57) ABSTRACT

Provided are methods of treating a KRAS mutant cancer in an individual. In certain embodiments, the methods include administering to an individual identified as having a KRAS mutant cancer a therapeutically effective amount of an agent that inhibits cardiotrophin-like cytokine factor 1 (CLCF1)-ciliary neurotrophic factor receptor (CNTFR) signaling. According to some embodiments, the KRAS mutant cancer is a KRAS mutant lung cancer, such as a KRAS mutant non-small cell lung cancer (NSCLC), e.g., a KRAS mutant lung adenocarcinoma (LUAD). Also provided are kits that find use, e.g., in practicing the methods of the present disclosure.

### CROSS-REFERENCE TO RELATED APPLICATIONS

This application claims the benefit of U.S. Provisional Patent Application No. 62/931,608, filed Nov. 6, 2019, and U.S. Provisional Patent Application No. 62/898,249, filed Sep. 10, 2019, which applications are incorporated herein by reference in their entireties.

### STATEMENT OF GOVERNMENT SUPPORT

This invention was made with Government support under contract no. R01 CA225103 awarded by the National Cancer Institute. The Government has certain rights in the invention.

### INTRODUCTION

Lung cancer is the leading cause of cancer-related death worldwide. The non-small cell lung cancer (NSCLC) subgroup accounts for 85-90% of cases and lung adenocarcinoma (LUAD) is the most common NSCLC histologic subtype. While approximately 30% of LUAD cases harbor a mutation in KRAS, these patients currently have few targeted therapeutic options. In LUAD subtypes characterized by EGFR or ALK alterations, small molecule inhibitors are effective, although rapid drug resistance remains a major limitation. Monoclonal antibody-based immunotherapy agents have also dramatically improved the available options and can have significant impact on survival for some patients. Despite these advances, there is a continued clinical need for innovative approaches to lung cancer treatment, especially those directed at mechanisms of oncogenesis currently not targeted by available agents.

Cancer is initiated and progresses within a microenvironment that is itself altered as a consequence of the tumorigenic process. Stromal cells in contact with cancer cells secrete growth factors and cytokines that may act directly by signaling to tumor cells or indirectly by recruiting other stromal components to promote tumor progression. An important aspect of this process is the expansion of cancer-associated fibroblasts (CAFs). CAFs are a diverse population of stromal cells with distinct characteristics in different tumors and tissues.

CAFs support the growth of cancer cells (e.g., lung cancer cells) in vivo by secretion of soluble factors that stimulate the growth of tumor cells. One such soluble factor is cardiotrophin-like cytokine factor 1 (CLCF1). CLCF1 belongs to the interleukin (IL)-6 family of structurally related hemato- and neuropoietic cytokines (IL-6, IL-11, ciliary neurotrophic factor (CNTF), leukemia inhibitory factor (LIF), oncostatin M (OSM), cardiotrophin-1 (CT-1)). CLCF1 produced by cells in the stroma is received as a growth signal by tumor cells expressing a receptor for this protein—the GPI-anchored CNTF receptor (CNTFR). Binding to membrane-bound or soluble CNTFR induces a heterodimer of the signal transducing β-receptors gp130 (a membrane-spanning 130-kDa glycoprotein) and LIF receptor (LIFR), which triggers intracellular signaling cascades such as the JAK STAT pathway and the MAPK/ERK pathway.

RAS family genes, including HRAS, KRAS and NRAS, are common oncogenes in human cancer, and encode extremely similar proteins made up of chains of 188 to 189 amino acids. The sequences and structural features of these three proteins are highly conserved, except for their carboxyl-terminal domains and post-translational lipid modifications. HRAS, KRAS and NRAS are regulated in a similar manner within the cell. The RAS genes encode monomeric GTPases that function as molecular switches in signal transduction pathways regulating cell proliferation, differentiation and survival in mammalian cells. Mutations that can constitutively activate RAS have been found in 20%˜25% of all human cancers. KRAS binds to GTP in its active state and possesses an intrinsic enzymatic activity which cleaves the terminal phosphate of the nucleotide, converting it to GDP. Upon conversion of GTP to GDP, KRAS is deactivated. The rate of conversion is usually slow, but can be increased dramatically by an accessory GTPase-activating protein (GAP). In turn, KRAS can bind to guanine nucleotide exchange factors (GEFs) (such as SOS), which force the release of bound nucleotide (GDP). GTP binding enables several residues, primarily in the switch I region (residues 30-40) and switch II region (residues 60-70), to adopt a conformation that permits KRAS effector proteins to bind; these switches are regulated by GAPs and GEFs. In mammalian cells, endogenous KRAS proteins are predominantly in the GDP state and activation is transient. However, the common oncogenic mutations in KRAS proteins interfere with GTP hydrolysis, resulting in proteins that remain in the active GTP state and continue to transmit signals to effector pathways. Thus, KRAS acts as a molecular on/off switch. Once it is turned on, it recruits and activates proteins necessary for the propagation of signaling of growth factors and other receptors, such as c-Raf and PI3K.

### SUMMARY

Provided are methods of treating a KRAS mutant cancer in an individual. In certain embodiments, the methods include administering to an individual identified as having a KRAS mutant cancer a therapeutically effective amount of an agent that inhibits cardiotrophin-like cytokine factor 1 (CLCF1)-ciliary neurotrophic factor receptor (CNTFR) signaling. According to some embodiments, the KRAS mutant cancer is a KRAS mutant lung cancer, such as a KRAS mutant non-small cell lung cancer (NSCLC), e.g., a KRAS mutant lung adenocarcinoma (LUAD). Also provided are kits that find use, e.g., in practicing the methods of the present disclosure.

### BRIEF DESCRIPTION OF THE FIGURES

FIG. 1 CLCF1 increases and CNTFR knockdown decreases tumor growth in human LUAD. Panel A: CLCF1 treatment for 72 h increases cell viability after serum starvation in LUAD cell lines A549, H23, and H358 in a concentration-dependent manner compared to untreated control. Panels B and D: Recombinant human CLCF1 phosphorylates STAT3 (Y705) in both a concentration- ([CLCF1]=10 nM) and (panels C and D) time-dependent (15 min after treatment) manner in A549, H23, and H358. Panel E: qRT-PCR measurements of CNTFR knockdown with shCNTFR or control shGFP (four biological replicates for each). ** P<0.01; *** P<0.001 using one-way analysis of variance (ANOVA). Data are represented as mean±S.D. Panel F: Proliferation of A549 after knockdown with indicated shRNAs. Two-way ANOVA. Panel G: Proliferation rates for LUAD cells after CNTFR knockdown at day 7 (four independent biological replicates with three technical replicates per group). One-way ANOVA. Panel H: Representative pictures of colony-formation assay in A549 and H23. Panel I: Quantification of colony number from panel H. Four independent biological replicates with three technical replicates per group. *** P<0.001 using one-way ANOVA. Data are represented as mean±S.D. Panel J: Representative images of spheres from cells grown in anchorage-independent conditions in A549 and H23. Panel K: Quantification of sphere number (three biological replicates). One-way ANOVA. Panel L: Tumor volume quantification of A549 xenografts with indicated shRNAs. * P<0.05 using two-way ANOVA. Data are represented as mean±S.E.M. Panel M: Tumor volume quantification of final time point in indicated LUAD cell line xenografts. Whiskers identify the maximum and minimum values; boxes indicate the 75^{th} and 25th percentile and line the median. One-way ANOVA. Panel N: Representative hematoxylin and eosin (H&E) staining and immunohistochemistry (IHC) for phospho-histone H3 (PH3) and cleaved caspase-3 (CC3) in A549 xenografts. Scale bars: 50 μm. Panel O: Quantification of PH3- and CC3-positive foci in A549, H23, and H2009 xenografts. * P<0.05; *** P<0.001 using one-way ANOVA. Data are represented as mean±S.E.M.

FIG. 2 Engineering a CNTFR receptor decoy using yeast display. Panel A: i) CNTFR transmits signal through the β receptors, gp130 and LIFR. ii) The β receptors become activated when CLCF1 complexes with CNTFR. iii) Soluble CNTFR allows gp130 and LIFR to heterodimerize even in cells lacking CNTFR expression. iv) Engineered soluble CNTFR (eCNTFR) that does not bind to the β receptors can function as an antagonist. Panel B: Schematic representation of yeast-displayed CNTFR and overlaid flow cytometry dot plot representing binding of yeast-displayed wtCNTFR to 10 nM (cyan) and 0 nM (red) CLCF1-His. Panel C: Flow cytometry histograms of the first CNTFR library and intermediate sorted population compared to wtCNTFR (WT), measuring binding to 0.5 nM CLCF1. Only the gated population of yeast expressing CNTFR is shown. Panel D: Binding curves of affinity matured yeast-displayed CNTFR variants with various concentrations of CLCF1 and the measured apparent K_{d} values. Panel E: Overlaid representative flow cytometry dot plots for sort 2 (red), sort 4 (blue), and sort 6 (orange) showing enrichment of non-LIFR binders. Panel F: Y177H and K178N isolated from negative screening against LIFR-Fc additively decreases LIFR-Fc binding. The measured apparent Kₐ values represent binding affinity toward CLCF1. Data are represented as mean (n=3 independent replicates)±S.D. * P<0.05; ** P<0.01; *** P<0.001.

FIG. 3 Characterization of eCNTFR constructs. Panel A: The 3D structure prediction of wtCNTFR (yellow) and eCNTFR (blue) was carried out with the Phyre 2 server (Protein Homology/analogY Recognition Engine V 2.0), showing locations of four mutations from affinity maturation (blue), two mutations to reduce LIFR binding (green), two mutations to reduce gp130 binding (magenta); the inset shows the aromatic cluster and conserved residues of CNTFR for cytokine binding (red) and mutations from affinity maturation (blue). Binding affinities of soluble wtCNTFR and eCNTFR constructs were compared for (panel B) CLCF1, (panel C) gp130-Fc and LIFR-Fc, (panel D) CNTF, and (panel E) mouse CLCF1. K_{d} values were calculated where appropriate. Data are represented as mean (n=3 independent replicates)±S.D. *** P<0.001 compared to the corresponding wtCNTFR construct. Panel F: Competition assay using ELISA to measure the ability of eCNTFR-Fc to block binding between wtCNTFR-Fc and CLCF1-His, LIFR-His, and gp130-His. Where LIFR-His and gp130-His were included CLCF1 (10 nM) was also added to induce complex formation. Panels G and H: eCNTFR-Fc inhibits STAT3 phosphorylation (Y705) in A549 and H23 cells. Panels I and J: eCNTFR-Fc inhibits CLCF1 induced cell survival in serum starved A549 and H23 cells. Data are represented as mean±S.D (n=3 independent replicates). ** P<0.01; *** P<0.001 compared to the corresponding non-eCNTFR-Fc treated control.

FIG. 4 Genotype specificity of eCNTFR-Fc in LUAD. Panel A: Cell line viability after treatment with 2.5 μM eCNTFR-Fc (three independent biological replicates with four technical replicates per group). Panel B: Western blot of A549 and H23 treated with serum, CLCF1, eCNTFR-Fc, CLCF1+eCNTFR-Fc, or eCNTFR-Fc+serum after 24 h serum starvation. Panel C: Quantification of western blot from panel B. Panel D: Ras-GTP levels assessed by Ras-GTP ELISA in cell lysates derived from A549 and H23 treated with serum, CLCF1, eCNTFR-Fc, CLCF1+eCNTFR-Fc, or eCNTFR-Fc+serum after 24 h serum starvation. Two biological replicates shown. Data are represented as mean (n=3 independent replicates)±S.D.

FIG. 5 Effect of eCNTFR-Fc in preclinical xenograft models. Panel A: Blood clearance and CLCF1 sequestration after intraperitoneal (i.p.) dosing of 10 mg/kg eCNTFR-Fc in non-tumor bearing NOD/SCID/gamma mice. Serum samples were collected post injection and unbound CLCF1 was measured by ELISA using eCNTFR-Fc as the capture agent. Vehicle-treated mice were used to determine baseline CLCF1 levels. Panel B: Tumor volume quantification of A549 xenografts [n=8 tumors except PBS (n=6 tumors at the last time point)]. * P<0.05; ** P<0.01; *** P<0.001; n.s.=not significant using two-way ANOVA. Panel C: Tumor volume quantification of final time point of A549 xenografts. Panel D: Waterfall plot showing tumor percent change from baseline for A549 xenografts. Panel E: Tumor volume quantification of patient-derived xenograft 727 (PDTX 727) model (n=16 tumors). Panel F: Tumor volume quantification of final time point of PDTX 727 and representative images of PDTX 727 tumors. Scale bars, 10 mm. Two-tailed unpaired Student's t-test. Panel G: Tumor volume quantification of final time point of PDTX models. Panel H: Representative H&E staining and IHC for phospho-histone H3 (PH3) and cleaved caspase-3 (CC3) from A549 xenografts. Scale bars, 50 μm. Panel I: Quantification of PH3- and CC3-positive foci. One-way ANOVA. Panel J: Representative H&E staining and IHC for PH3 and CC3 from PDTX xenografts. Scale bars, 50 μm. Panel K: Quantification of PH3- and CC3-positive foci. Two-tailed unpaired Student's t-test. Panel L: Representative IHC for phospho-ERK (P-ERK) and Phospho-S6RP (P-S6) in A549 xenografts and (Panel M) PDTX. Panel N: Western blot of A549 xenografts. Panel O: Quantification of western blot. Data are represented as mean±S.E.M.

FIG. 6 Effect of eCNTFR-Fc in an autochthonous KRAS-driven genetically-engineered mouse model. Panel A: Representative 2D axial microCT (μCT) images, cross-section of mouse lungs at cervical vertebra 8 from KRAS^{G12D}/P53^{f/f} (KRAS; P53) mice treated 3 times/week with PBS or eCNTFR-Fc (10 mg/kg) for 4 weeks (Day 28) starting at 8 weeks post-delivery of 5×10⁶ pfu of adenovirus expressing Cre (Day 0). Red outline surrounds the heart and red arrow identifies representative tumor nodule. Panel B: Quantification of μCT tumor burden using ImageJ software. Arbitrary units (A.U.). Panel C: Representative H&E images of lungs 28 days after treatment initiation. Scale bars, 1 mm. Panel D: Effect of treatment on tumor burden (%) and (panel E) tumor foci. *** P<0.001 using two-tailed unpaired Student's t-test. Data are represented as mean±S.E.M. Panel F: Representative IHC for PH3 and CC3 from the GEM model. Panel G: Quantification of PH3- and CC3-positive foci. *** P<0.001 using two-tailed unpaired Student's t-test. Panel H: Representative IHC for phospho-ERK (P-ERK), Phospho-S6RP (P-S6), and phopho-STAT3 (P-STAT3) 28 days after treatment initiation. Panel I: Kaplan-Meier analysis of survival to ethical endpoint of mice from the same experiment (n=11 mice per group). Log-rank test. Panel J: CLCF1 ELISA performed on patient plasma samples and normal controls. Mutation of interest=KRAS G12C, KRAS G12V, and EGFR Mutant/KRAS wt.

FIG. 7 CLCF1 expression across 40 cancer types. CLCF1 expression is plotted as log₂ normalized transcripts per million (TPM) on the x-axis. Data was downloaded from publicly available repositories (TCGA). Figure is plotted as log₂ (TPM+1), ranked by mean. Blue line denotes the 75% quantile of CLCF1 expression across all samples. Abbreviations: The Cancer Genome Atlas, TCGA.

### DETAILED DESCRIPTION

Before the methods and kits of the present disclosure are described in greater detail, it is to be understood that the methods and kits are not limited to particular embodiments described, as such may, of course, vary. It is also to be understood that the terminology used herein is for the purpose of describing particular embodiments only, and is not intended to be limiting, since the scope of the methods and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and kits. The upper and lower limits of these smaller ranges may independently be included in the smaller ranges and are also encompassed within the methods and kits, subject to any specifically excluded limit in the stated range. Where the stated range includes one or both of the limits, ranges excluding either or both of those included limits are also included in the methods and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and kits belong. Although any methods and kits similar or equivalent to those described herein can also be used in the practice or testing of the methods and kits, representative illustrative methods and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and kits, which are, for clarity, described in the context of separate embodiments, may also be provided in combination in a single embodiment. Conversely, various features of the methods and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods of treating a KRAS mutant cancer in an individual. In certain embodiments, the methods include administering to an individual identified as having a KRAS mutant cancer a therapeutically effective amount of an agent that inhibits cardiotrophin-like cytokine factor 1 (CLCF1)-ciliary neurotrophic factor receptor (CNTFR) signaling. The present disclosure is based in part on the surprising findings demonstrated for the first time herein that targeting the CLCF1-CNTFR signaling axis in KRAS mutant cancers provides a significant anti-tumor effect. Details regarding embodiments of the methods of the present disclosure will now be described.

KRAS Mutant Cancers

As summarized above, the agent is administered to an individual identified as having a KRAS mutant cancer. By “KRAS mutant cancer” is meant a cancer in which the initiation and/or maintenance are/is dependent, at least in part, on one or more mutations in the gene that encodes KRAS (human: UniProtKB—P01116). In certain embodiments, the one or more KRAS mutations constitutively activate KRAS and subsequently its downstream Raf/MEK/ERK1/2 and/or PI3K/PIP3/AKT survival pathways in cancer cells of the KRAS mutant cancer.

As used herein, a “cancer” comprises one or more cancer cells, where by “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like.

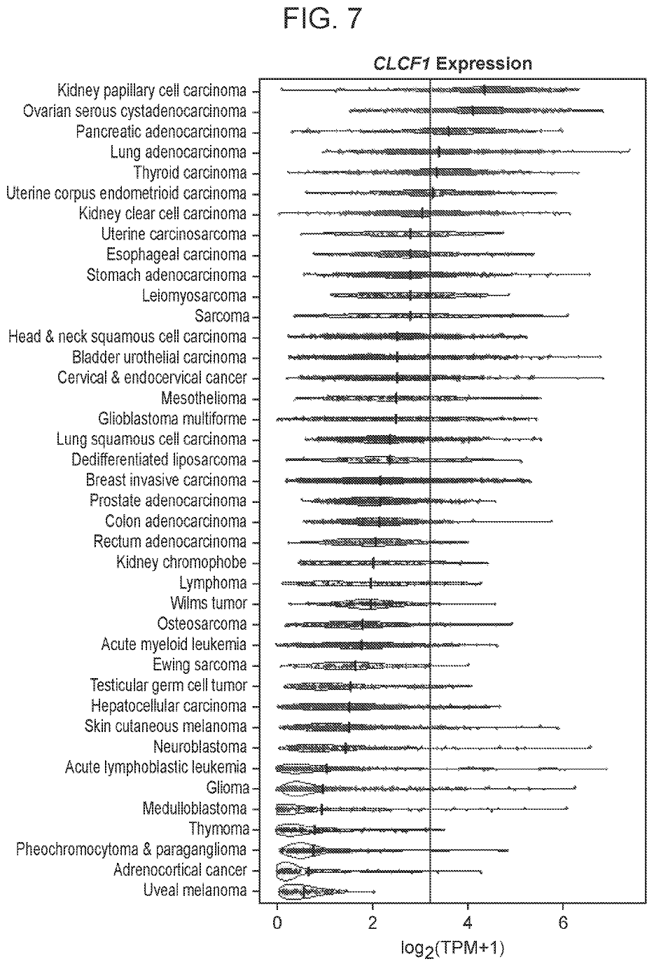

In certain embodiments, the individual has a KRAS mutant cancer characterized by the presence of a solid tumor, a semi-solid tumor, a primary tumor, a metastatic tumor, a liquid tumor (e.g., a leukemia or lymphoma), and/or the like. According to some embodiments, the individual has a KRAS mutant cancer selected from breast cancer, glioblastoma, neuroblastoma, head and neck cancer, gastric cancer, ovarian cancer, skin cancer (e.g., basal cell carcinoma, melanoma, or the like), lung cancer, colorectal cancer, prostate cancer, glioma, bladder cancer, endometrial cancer, kidney cancer, leukemia (e.g., T-cell acute lymphoblastic leukemia (T-ALL), acute myeloid leukemia (AML), etc.), liver cancer (e.g., hepatocellular carcinoma (HCC), such as primary or recurrent HCC), a B-cell malignancy (e.g., non-Hodgkin lymphomas (NHL), chronic lymphocytic leukemia (CLL), follicular lymphoma, mantle cell lymphoma, diffuse large B-cell lymphoma, and the like), pancreatic cancer, thyroid cancer, any combinations thereof, and any sub-types thereof. In certain embodiments, the individual's KRAS mutant cancer is a human pancreatic ductal adenocarcinoma (PDAC), non-small cell lung cancer, colorectal cancer, and/or biliary cancer. In any embodiments of the present disclosure, the KRAS mutant cancer may be one characterized by the presence of CLCF1 in the tumor microenvironment. Non-limiting examples of cancers that exhibit expression of CLCF1 are shown in FIG. 7.

In certain embodiments, the KRAS mutant cancer is a KRAS mutant lung cancer. Non-limiting examples of KRAS mutant lung cancers that may be treated according to the methods of the present disclosure include KRAS mutant small cell lung cancers (SCLC) and KRAS mutant non-small cell lung cancers (NSCLC). When the individual has a KRAS mutant NSCLC, in some embodiments, the individual has a KRAS mutant lung adenocarcinoma (LUAD).

According to some embodiments, the KRAS mutant cancer is a KRAS mutant pancreatic cancer. A non-limiting example of a KRAS mutant pancreatic cancer that may be treated according to the methods of the present disclosure include KRAS mutant human pancreatic ductal adenocarcinoma (PDAC).

The KRAS mutant cancer may be characterized by any of a variety of one or more KRAS mutations. Non-limiting examples of KRAS mutations include insertions, deletions, one or more amino acid substitution-inducing mutations, and/or the like, in the gene encoding KRAS. According to some embodiments, the KRAS mutant cancer comprises an amino acid substitution at one or more positions of human KRAS (UniProtKB—P01116), the amino acid sequence of which is provided in Table 1 below. In certain embodiments, the KRAS mutant cancer comprises an amino acid substitution at one or more of positions 12, 13, 61, 117, and 146 of human KRAS. By way of example, the KRAS mutant cancer may comprise one or more of the following amino acid substitutions in human KRAS: G12A, G12C, G12D, G12R, G12S, G12V, G13D, Q61H, Q61K, K117N and A146T. According to some embodiments, the KRAS mutant cancer comprises a substitution at position 12 of KRAS. When the KRAS mutant cancer comprises a substitution at position 12, the KRAS mutant cancer may comprise, or consist of, an amino acid substitution selected from G12A, G12C, G12D, G12R, G12S, and G12V (where “consist of” as used in this context means the amino acid substitution is the only KRAS mutation in the KRAS mutant cancer). When the KRAS mutant cancer comprises a substitution at position 12, the KRAS mutant cancer may comprise, or consist of, an amino acid substitution selected from G12A, G12C, G12D, G12S, and G12V. In certain embodiments, the KRAS mutant cancer comprises or consists of the amino acid substitution G12A. In certain embodiments, the KRAS mutant cancer comprises or consists of the amino acid substitution G12C. According to some embodiments, the KRAS mutant cancer comprises or consists of the amino acid substitution G12D. In certain embodiments, the KRAS mutant cancer comprises or consists of the amino acid substitution G12R. According to some embodiments, the KRAS mutant cancer comprises or consists of the amino acid substitution G12S. In certain embodiments, the KRAS mutant cancer comprises or consists of the amino acid substitution G12V.

TABLE 1
Wild-type human KRAS amino acid sequence (UniProtKB - P01116)

| | |
|---|---|
| Wild-type human | MTEYKLVVVGAGGVGKSALTIQLIQNHFVDEYDPTIEDSYRKQ |
| KRAS amino acid | VVIDGETCLLDILDTAGQEEYSAMRDQYMRTGEGFLCVFAINN |
| sequence | TKSFEDIHHYREQIKRVKDSEDVPMVLVGNKCDLPSRTVDTK |
| (UniProtKB - P01116) | QAQDLARSYGIPFIETSAKTRQRVEDAFYTLVREIRQYRLKKIS |
| (SEQ ID NO: 1) | KEEKTPGCVKIKKCIIM |

As used herein, the agent being administered to an individual “identified” as having a KRAS mutant cancer means that the agent is administered to the individual based at least in part on knowledge, prior to the administration, that the individual has a KRAS mutant cancer or subtype thereof, e.g., knowledge that the individual's cancer is a KRAS mutant cancer comprising or consisting of an amino acid substitution at position 12 of KRAS, such as G12A, G12C, G12D, G12R, G12S, G12V, G13D, Q61H, Q61K, K117N, or A146T.

In certain embodiments, the methods of the present disclosure further include identifying the individual as having a KRAS mutant cancer. Identifying the individual as having a KRAS mutant cancer may include, e.g., receiving and reviewing a report indicating that the individual's cancer is a KRAS mutant cancer or subtype thereof, e.g., receiving and reviewing a report indicating that the individual's cancer is a KRAS mutant cancer comprising or consisting of an amino acid substitution at position 12 of KRAS, such as G12A, G12C, G12D, G12R, G12S, G12V, G13D, Q61H, Q61K, K117N, or A146T.

According to some embodiments, identifying the individual as having a KRAS mutant cancer comprises determining that the individual's cancer is a KRAS mutant cancer. A variety of approaches may be employed to determine that the individual's cancer is a KRAS mutant cancer, non-limiting examples of which include assaying a biopsy sample of the cancer for one or more KRAS mutations. Suitable assays include, but are not limited to, sequencing the gene or mRNA transcripts encoding KRAS in cancer cells of the individual (e.g., using an available nucleic acid sequencing system from Illumina, Oxford Nanopore Technologies, Pacific Biosciences, or the like); performing PCR using mutation-specific amplification primers that interrogate the gene or mRNA transcript encoding KRAS for one or more mutations of interest; using an antibody-based assay that employs one or more antibodies that specifically bind to one or more particular mutant KRAS proteins; and/or any other suitable assay for determining whether the individual's cancer comprises one or more KRAS mutations.

In certain embodiments, the agent is only administered to an individual identified as having a particular type of KRAS mutant cancer. For example, according to some embodiments, the agent is only administered to an individual identified as having a KRAS mutant cancer comprising an amino acid substitution at position 12 of KRAS, wherein numbering is as in SEQ ID NO:1. In certain embodiments, the agent is only administered to an individual identified as having a KRAS mutant cancer comprising an amino acid substitution selected from the group consisting of: G12A, G12C, G12D, G12S, and G12V. According to some embodiments, the agent is only administered to an individual identified as having a KRAS mutant cancer comprising an amino acid substitution selected from the group consisting of: G12A, G12C, G12D, G12S , and G12V, and also only when the individual has been identified as having a plasma CLCF1 concentration above a threshold plasma CLCF1 concentration. As used herein, “only administered” means the agent is not administered to the individual unless the individual meets the specified criteria, e.g., type of KRAS mutation(s), plasma CLCF1 concentration, and/or the like.

The individual having a KRAS mutant cancer may vary. In certain embodiments, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). According to some embodiments, the individual is a human. In certain embodiments, the individual is an animal model (e.g., a mouse model, a primate model, or the like) of a cancer, e.g., a KRAS mutant cancer.

Agents

The agent administered to the individual identified as having a KRAS mutant cancer may be any agent that that inhibits (e.g., decreases or blocks) cardiotrophin-like cytokine factor 1 (CLCF1)-ciliary neurotrophic factor receptor (CNTFR) signaling. Agents that may be employed include small molecules, protein-based agents (e.g., peptides, antibodies, engineered ligands, engineered receptors, etc.), and/or the like. The agent may be detectably labeled, e.g., with an in vivo imaging agent, or the like. The agent may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the agent upon administration to the subject.

By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less.

According to some embodiments, the agent is an antibody. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the target, including, but not limited to, Fv, single chain Fv (scFv), Fab, F(ab′)₂, Fab′, (scFv′)₂, and diabodies; chimeric antibodies; monoclonal antibodies, human antibodies, humanized antibodies (e.g., humanized whole antibodies, humanized antibody fragments, etc.); and fusion proteins including an antigen-binding portion of an antibody and a non-antibody protein or fragment thereof, e.g., an antibody Fc region or fragment thereof.

Agents that Bind CNTFR

In certain embodiments, the methods comprise administering an agent that specifically binds CNTFR and inhibits signaling through CNTFR. Such an agent may be, e.g., a small molecule, an antibody, a CNTFR ligand (e.g., an engineered CNTFR ligand), or the like. A non-limiting example of such an agent is one that specifically binds CNTFR and inhibits interaction between CNTFR and its ligands, e.g., CLCF1, CNTF, NP, and/or the like. CNTFR is the ligand-specific component of a tripartite receptor for ciliary neurotrophic factor (CNTF), as well as other ligands such as cardiotrophin-like cytokine factor 1 (CLCF1) and neuropoietin (NP). Binding of wild-type ligand to CNTFR recruits the transmembrane components of the receptor, gp130 and leukemia inhibitory factor receptor (LIFR), facilitating signal transduction. Wild-type amino acid sequences for human CNTFR, CNTF, CLCF1 and NP are provided in Table 2.

TABLE 2
Wild-Type Human CNTFR and CNTFR Ligand Amino Acid Sequences

| | Amino Acid Sequence |
|---|---|
| Wild-Type Human | MAAPVPWACCAVLAAAAAVVYAQRHSPQEAPHVQYERLGSDVTLPCG |
| CNTFR | TANWDAAVTWRVNGTDLAPDLLNGSQLVLHGLELGHSGLYACFHRDS |
| (SEQ ID NO: 2) | WHLRHQVLLHVGLPPREPVLSCRSNTYPKGFYCSWHLPTPTYIPNTFN |
| | VTVLHGSKIMVCEKDPALKNRCHIRYMHLFSTIKYKVSISVSNALGHNAT |
| | AITFDEFTIVKPDPPENVVARPVPSNPRRLEVTWQTPSTWPDPESFPLK |
| | FFLRYRPLILDQWQHVELSDGTAHTITDAYAGKEYIIQVAAKDNEIGTWS |
| | DWSVAAHATPWTEEPRHLTTEAQAAETTTSTTSSLAPPPTTKICDPGEL |
| | GSGGGPSAPFLVSVPITLALAAAAATASSLLI |
| Wild-Type Human | MDLRAGDSWGMLACLCTVLWHLPAVPALNRTGDPGPGPSIQKTYDLT |
| CLCF1 | RYLEHQLRSLAGTYLNYLGPPFNEPDFNPPRLGAETLPRATVDLEVWR |
| (SEQ ID NO: 3) | SLNDKLRLTQNYEAYSHLLCYLRGLNRQAATAELRRSLAHFCTSLQGLL |
| | GSIAGVMAALGYPLPQPLPGTEPTWTPGPAHSDFLQKMDDFWLLKELQ |
| | TWLWRSAKDFNRLKKKMQPPAAAVTLHLGAHGF |
| Wild-Type Human | MAFTEHSPLTPHRRDLCSRSIWLARKIRSDLTALTESYVKHQGLNKNINL |
| CNTF | DSADGMPVASTDQWSELTEAERLQENLQAYRTFHVLLARLLEDQQVHF |
| (SEQ ID NO: 4) | TPTEGDFHQAIHTLLLQVAAFAYQIEELMILLEYKIPRNEADGMPINVGD |
| | GGLFEKKLWGLKVLQELSQWTVRSIHDLRFISSHQTGIPARGSHYIANN |
| | KKM |
| Wild-Type Human | MYCLLATPLCLLSLLLPPLSPAAPISPSEPIGQAYSLALYMQKNTSALLQT |
| NP | YLQHQGSPFSDPGFSAPELQLSTLPSAAVSFKTWHAMEDAERLSRAQ |
| (SEQ ID NO: 5) | GAFLALTQHLQLVGDDQSYLNPGSPILLAQLGAARLRAQGLLGNMAAIM |
| | TALGLPIPPEEDTLGFVPFGASAFERKCRGYIVTREYGHWTDRAVRDLA |
| | LLKAKYSA |

According to some embodiments, the agent specifically binds CNTFR or a ligand-CNTFR complex subunit (e.g., gp130 or LIFR) and inhibits interaction between CNTFR and the ligand-CNTFR complex subunit.

In certain embodiments, the agent is an engineered CNTFR ligand. As used herein, an “engineered CNTFR ligand” is a polypeptide that binds to CNTFR and is a variant of a wild-type CNTFR ligand, such as a variant CNTF ligand, a variant CLCF1 ligand, or a variant NP ligand. By “variant” is meant the engineered CNTFR ligand includes one or more mutations relative to the corresponding wild-type CNTFR ligand. For example, an engineered CNTF ligand may include one or more mutations relative to wild-type CNTF, a CLCF1 ligand of the present disclosure may include one or more mutations relative to wild-type CLCF1, etc. As used throughout the present disclosure, a “mutation” or “mutations” may include one or more amino acid substitutions, one or more amino acid deletions (e.g., truncations), one or more amino acid insertions, or any combination thereof, in the polypeptide relative to the corresponding wild-type polypeptide.

According to some embodiments, when the agent is an engineered CNTFR ligand, the agent is an engineered CNTFR ligand that exhibits increased binding affinity for CNTFR relative to the corresponding wild-type CNTFR ligand. In certain embodiments, when the agent is an engineered CNTFR ligand, the agent is an engineered CNTFR ligand that results in reduced binding affinity of gp130, LIFR, or both, for a complex comprising the engineered CNTFR ligand and CNTFR, relative to the binding affinity for a complex comprising the corresponding wild-type CNTFR ligand and CNTFR. According to some embodiments, when the agent is an engineered CNTFR ligand, the agent is an engineered CNTFR ligand that exhibits increased binding affinity for CNTFR relative to the corresponding wild-type CNTFR ligand and results in reduced binding affinity of gp130, LIFR, or both, for a complex comprising the engineered CNTFR ligand and CNTFR, relative to the binding affinity for a complex comprising the corresponding wild-type CNTFR ligand and CNTFR. By “increased binding affinity” or “greater binding affinity” is meant that the CNTFR ligand exhibits tighter binding (as indicated by a lower K_{D} value) to CNTFR as compared to the corresponding wild-type CNTFR ligand. By way of example, in certain aspects, when the CNTFR ligand is a variant CLCF1 ligand, the binding affinity of the CLCF1 ligand for CNTFR has a K_{D} value that is 20 nM or less.

As used herein, a first molecule “specifically binds” to a second molecule if it binds to or associates with the second molecule with an affinity or Kₐ (that is, an equilibrium association constant of a particular binding interaction with units of 1/M) of, for example, greater than or equal to about 10⁵ M⁻¹. In certain embodiments, the first molecule binds to the second molecule with a Kₐ greater than or equal to about 10⁶ M⁻¹, 10⁷ M⁻¹, 10⁸ M⁻¹, 10⁹ M⁻¹, 10¹⁰ M⁻¹, 10¹¹M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In certain aspects, specific binding means binding to the target molecule with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹M, or 10⁻¹² M or less. The binding affinity of the first molecule for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In certain embodiments, the CNTFR ligand that exhibits increased binding affinity for CNTFR relative to the corresponding wild-type CNTFR ligand is a CLCF1 ligand (which may be referred to as a “variant CLCF1” or an “engineered CLCF1”). In some embodiments, such a CLCF1 ligand may include one or more mutations at amino acid positions 86, 96, 148, 169, 180, or any combination thereof, wherein numbering is as in SEQ ID NO:3. By way of example, such a CLCF1 ligand may include one or more mutations selected from L86F, Q96R, H148R, W169L, K180R, and any combination thereof, relative to a CLCF1 ligand having the amino acid sequence set forth in SEQ ID NO:3. Non-limiting examples of CLCF1 variants exhibiting increased binding affinity for CNTFR, as well as strategies for identifying additional such variants, are described in U.S. Ser. No. 16/465,726, the disclosure of which is incorporated herein by reference in its entirety for all purposes.

In some embodiments, the CNTFR ligand results in reduced binding affinity of gp130 for a complex comprising the CNTFR ligand and CNTFR. In some embodiments, such a ligand is a CLCF1 ligand that includes one or more mutations at amino acid positions 22, 169, 180, or any combination thereof, wherein numbering is as in SEQ ID NO:3. By way of example, such a CLCF1 ligand may include one or more mutations selected from Y22C, W169L, K180R, and any combination thereof, relative to a CLCF1 ligand having the amino acid sequence set forth in SEQ ID NO:3. Non-limiting examples of CLCF1 variants resulting in reduced binding affinity of gp130 for a complex including the CLCF1 variant and CNTFR, as well as strategies for identifying additional such variants, are described in U.S. Ser. No. 16/465,726, the disclosure of which is incorporated herein by reference in its entirety for all purposes.

In some embodiments, in a direct binding assay, an equilibrium binding constant (K_{D}) may be measured using a CNTFR ligand, gp130, or LIFR conjugated to a fluorophore or radioisotope, or a CNTFR ligand, gp130, or LIFR that contains an N- or C-terminal epitope tag for detection by a labeled antibody. If labels or tags are not feasible or desired, a competition binding assay can be used to determine the half-maximal inhibitory concentration (IC₅₀), the amount of unlabeled CNTFR ligand, gp130, or LIFR at which 50% of the maximal signal of the labeled competitor is detectable. A K_{D} value can then be calculated from the measured IC₅₀ value.

The amino acid sequences of two non-limiting examples of CNTFR ligands of the present disclosure are provided in Table 3 below.

TABLE 3
Amino Acid Sequences of Example Engineered CNTFR Ligands

| | Amino Acid Sequence |
|---|---|
| Example CNTFR Ligand | LNRTGDPGPGPSIQKTYDLTRYLEHQLRSLAGTYLNYL |
| (CLCF1 variant) | GPPFNEPDFNPPRLGAETLPRATVDLEVWRSLNDKLR |
| (SEQ ID NO: 6) | LTQNYEAYSHFLCYLRGLNRRAATAELRRSLAHFCTSL |
| (L86F, Q96R, H148R) | QGLLGSIAGVMAALGYPLPQPLPGTEPTWTPGPARSD |
| | FLQKMDDFWLLKELQTWLWRSAKDFNRLKKKMQPPA |
| | AAVTLHLGAHGF |
| Example CNTFR Ligand | LNRTGDPGPGPSIQKTYDLTRCLEHQLRSLAGTYLNYL |
| (CLCF1 variant) | GPPFNEPDFNPPRLGAETLPRATVDLEVWRSLNDKLR |
| (SEQ ID NO: 7) | LTQNYEAYSHFLCYLRGLNRRAATAELRRSLAHFCTSL |
| (Y22C, L86F, Q96R, H148R, | QGLLGSIAGVMAALGYPLPQPLPGTEPTWTPGPARSD |
| F151A, K154A, W169L, K180R) | ALQAMDDFWLLKELQTWLLRSAKDFNRLKRKMQPPA |
| | AAVTLHLGAHGF |

The example CNTFR ligands in Table 3 are engineered CLCF1 variants. Both variants exhibit increased binding affinity for CNTFR relative to wild-type CLCF1. The second variant additionally results in reduced binding affinity of gp130 and LIFR to a complex that includes this variant and CNTFR. In some embodiments, the CNTFR ligand is a CNTFR ligand presented in Table 3. In some embodiments, such a CNTFR ligand is present in a fusion protein (e.g., fused to an Fc domain), conjugate (e.g., conjugated to PEG, a drug, and/or the like), or combination thereof.

In certain embodiments, a CNTFR ligand of the present disclosure binds to CNTFR and has 70% or greater, 75% or greater, 80% or greater, 85% or greater, 90% or greater, 95% or greater, 99% or greater, or 100% amino acid sequence identity to a CNTFR ligand presented in Table 3. In some embodiments, such a CNTFR ligand is present in a fusion protein (e.g., fused to an Fc domain), conjugate (e.g., conjugated to PEG, a drug, and/or the like), or combination thereof.

In certain aspects, the CNTFR ligand is a CLCF1 variant that binds to CNTFR and includes an amino acid substitution selected from L86F, Q96R, H148R, and any combination thereof, where the CLCF1 variant includes 70% or greater, 75% or greater, 80% or greater, 85% or greater, 90% or greater, 95% or greater, 99% or greater, or 100% amino acid sequence identity to the amino acid sequence set forth in SEQ ID NO:6. In some embodiments, such a CNTFR ligand is present in a fusion protein (e.g., fused to an Fc domain), conjugate (e.g., conjugated to PEG, a drug, and/or the like), or combination thereof.

In certain aspects, the CNTFR ligand is a CLCF1 variant that binds to CNTFR and includes an amino acid substitution selected from Y22C, L86F, Q96R, H148R, F151A, K154A, W169L, K180R, and any combination thereof, where the CLCF1 variant includes 70% or greater, 75% or greater, 80% or greater, 85% or greater, 90% or greater, 95% or greater, 99% or greater, or 100% amino acid sequence identity to the amino acid sequence set forth in SEQ ID NO:7. In some embodiments, such a CNTFR ligand is present in a fusion protein (e.g., fused to an Fc domain), conjugate (e.g., conjugated to PEG, a drug, and/or the like), or combination thereof.

Agents that Bind CLCF1

In certain embodiments, the agent that inhibits CLCF1-CNTFR signaling is an agent that specifically binds CLCF1 and inhibits signaling through CNTFR. Such an agent may be, e.g., a small molecule, an antibody, a CLCF1 receptor (e.g., an engineered soluble CLCF1 receptor), or the like. A non-limiting example of such an agent is one that specifically binds CLCF1 and inhibits interaction between CLCF1 and CNTFR.

According to some embodiments, an agent that specifically binds CLCF1 is a soluble CNTFR polypeptide. By “soluble CNTFR polypeptide” is meant a CNTFR polypeptide that is not integrated into a cell membrane. The wild-type human CNTFR amino acid sequence (UniProtKB—P26992) is provided in Table 4 below.

TABLE 4
Wild-Type Human CNTFR Amino Acid Sequence (Non-Soluble)

| | Amino Acid Sequence |
|---|---|
| Wild-Type Human | MAAPVPWACCAVLAAAAAVVYAQRHSPQEAPHVQYERLGSDVTLPCG |
| CNTFR (non- | TANWDAAVTWRVNGTDLAPDLLNGSQLVLHGLELGHSGLYACFHRDS |
| soluble) | WHLRHQVLLHVGLPPREPVLSCRSNTYPKGFYCSWHLPTPTYIPNTFN |
| (SEQ ID NO: 8) | VTVLHGSKIMVCEKDPALKNRCHIRYMHLFSTIKYKVSISVSNALGHNAT |
| | AITFDEFTIVKPDPPENVVARPVPSNPRRLEVTWQTPSTWPDPESFPLK |
| | FFLRYRPLILDQWQHVELSDGTAHTITDAYAGKEYIIQVAAKDNEIGTWS |
| | DWSVAAHATPWTEEPRHLTTEAQAAETTTSTTSSLAPPPTTKICDPGEL |
| | GSGGGPSAPFLVSVPITLALAAAAATASSLLI |

According to certain embodiments, the soluble CNTFR polypeptide is not integrated into a cell membrane by virtue of the polypeptide having one or more solubility-conferring mutations. The one or more solubility-conferring mutations may be located in any suitable region(s) of the CNTFR polypeptide. In certain aspects, the soluble CNTFR polypeptide includes one or more solubility-conferring mutations in the domain that anchors wild-type CNTFR to the cell membrane. This domain contains a lipidation site (S342) that is post-translationally modified with glycosylphosphatidylinositol (GPI), which anchors the protein to the cell membrane. The wild-type human CNTFR domain that anchors CNTFR to the cell membrane can be defined as consisting of amino acids 343-372, wherein numbering is as in SEQ ID NO:8 (underlined in Table 4). Under certain conditions, this portion of CNTFR is enzymatically modified to release CNTFR from the cell membrane. According to some embodiments, a soluble CNTFR polypeptide of the present disclosure includes a substitution mutation at S342 that precludes post-translational modification with GPI, thereby conferring solubility. Wild-type human CNTFR also includes a signal peptide consisting of amino acids 1-22 of SEQ ID NO:8 (underlined in Table 4).

According to certain embodiments, the CNTFR domain that anchors CNTFR to the cell membrane includes one or more amino acid substitutions that result in the CNTFR polypeptide losing its ability to be anchored to a cell membrane, thereby conferring solubility. Alternatively, or additionally, the soluble CNTFR polypeptide may include a truncation (e.g., in the CNTFR domain that anchors CNTFR to the cell membrane) that results in the CNTFR polypeptide losing its ability to be anchored to a cell membrane, thereby conferring solubility. In certain aspects, the soluble CNTFR polypeptide lacks the CNTFR domain that anchors CNTFR to the cell membrane. For example, the soluble CNTFR polypeptide may lack amino acids 343-372 set forth in SEQ ID NO:8.

In addition to optionally including one or more solubility-conferring mutations, a soluble CNTFR polypeptide of the present disclosure may include one or more mutations that confer one or more other desirable properties upon the polypeptide. Other desirable properties of interest include, but are not limited to, greater binding affinity for CLCF1, altered (e.g., greater) specificity for CLCF1 as compared to one or more other CNTFR ligands, altered (e.g., reduced) binding affinity for a ligand-CNTFR complex subunit (e.g., gp130, LIFR, and/or the like), relative to a wild-type CNTF receptor, e.g., a receptor having the amino acid sequence set forth in SEQ ID NO:8 or a mature form thereof.

By “greater binding affinity” or “increased binding affinity” is meant that the soluble CNTFR polypeptide exhibits tighter binding (as indicated by a lower K_{D} value) to CLCF1 as compared to a wild-type CNTF receptor. By “lower binding affinity” or “reduced binding affinity” is meant that the soluble CNTFR polypeptide exhibits less tight binding (as indicated by a higher K_{D} value) to a molecule (e.g., a ligand-CNTFR complex subunit such as LIFR, gp130, or both) as compared to a wild-type CNTF receptor.

Methods are available for measuring the binding affinity of a CNTFR ligand-binding agent (e.g., a soluble CNTFR polypeptide) to a molecule of interest, e.g., CLCF1, a ligand-CNTFR complex subunit such as LIFR, gp130, or the like. For example, surface plasmon resonance (SPR) technology (e.g., using a BIAcore™ 2000 instrument), KinExA® kinetic exclusion assay (Sapidyne Instruments), Bio-Layer Interferometry (BLI) technology (e.g., ForteBio Octet®), or other similar assay/technology may be employed to determine whether a CNTFR ligand-binding agent exhibits a desired binding affinity. Suitable approaches for measuring binding affinity in the context of the present disclosure include, e.g., those described in Hunter, S. A. and Cochran, J. R. (2016) Methods Enzymol. 580:21-44.

In some embodiments, in a direct binding assay, an equilibrium binding constant (K_{D}) may be measured using a CNTFR polypeptide conjugated to a fluorophore or radioisotope, or a CNTFR polypeptide that contains an N- or C-terminal epitope tag for detection by a labeled antibody. If labels or tags are not feasible or desired, a competition binding assay can be used to determine the half-maximal inhibitory concentration (IC₅₀), the amount of unlabeled CNTFR polypeptide at which 50% of the maximal signal of the labeled competitor is detectable. A K_{D} value can then be calculated from the measured IC₅₀ value.

As summarized above, in certain aspects, a soluble CNTFR polypeptide of the present disclosure includes one or more mutations that alters (e.g., reduces) the binding affinity of the soluble CNTFR polypeptide for a CLCF1-CNTFR complex subunit relative to a wild-type CNTF receptor, e.g., a receptor having the amino acid sequence set forth in SEQ ID NO:8 or a mature form thereof. By “CLCF1-CNTFR complex subunit” is meant a protein that associates with wild-type CNTFR upon binding of CNTFR to CLCF1. Non-limiting examples of ligand-CNTFR complex subunits include LIFR and gp130. In certain embodiments, the one or more mutations reduces the binding affinity of the soluble CNTFR polypeptide for LIFR, gp130, or both. The one or more mutations may prevent the soluble CNTFR polypeptide from acting as an agonist upon binding to CLCF1 to reduce CNTFR-mediated signaling (e.g., to reduce cell proliferation).

In some embodiments, when the soluble CNTFR polypeptide exhibits reduced binding affinity for a CLCF1-CNTFR complex subunit, the binding affinity of the soluble CNTFR polypeptide has a K_{D} value that is 100 nM or greater in the presence of 10 nM of CLCF1.

In certain aspects, the soluble CNTFR polypeptide has reduced binding affinity for LIFR and includes a mutation (e.g., an amino acid substitution) at amino acid position 177, 178, or both, relative to a CNTFR polypeptide having the amino acid sequence set forth in SEQ ID NO:8. An example mutation at position 177 is Y177H. Another example mutation at position 177 is Y177A. An example mutation at position 178 is K178N. Another example mutation at position 178 is K178A. Such mutations result in the soluble CNTFR polypeptide being an inhibitor of CNTFR signaling, whereas a soluble CNTFR polypeptide having unaltered affinity for CLCF1-CNTFR complex subunits acts as an agonist by virtue of its ability to recruit, e.g., LIFR and gp130 upon binding CLCF1. In certain aspects, a soluble CNTFR polypeptide of the present disclosure includes the mutations Y177H and K178N, or the mutations Y177A and K178A, or the mutations Y177H and K178A, or the mutations Y177A and K178N.

According to certain embodiments, the soluble CNTFR polypeptide has reduced binding affinity for gp130 and includes a mutation (e.g., an amino acid substitution) at amino acid position 268, 269, or both, relative to a CNTFR polypeptide having the amino acid sequence set forth in SEQ ID NO:8. An example mutation at position 268 is T268A. An example mutation at position 269 is D269A. In certain aspects, the soluble CNTFR polypeptide includes the mutations T268A and D269A.

As summarized above, the soluble CNTFR polypeptide may include one or more mutations that alters (e.g., increases) the binding affinity and/or specificity of the soluble CNTFR polypeptide for CLCF1 relative to a wild-type CNTF receptor, e.g., a receptor having the amino acid sequence set forth in SEQ ID NO:8 or a mature form thereof. According to certain embodiments, when the soluble CNTFR polypeptide exhibits increased binding affinity for CLCF1, the binding affinity of the soluble CNTFR polypeptide for CLCF1 has a K_{D} value that is 10 nM or less.

In some embodiments, the soluble CNTFR polypeptide includes one or more mutations that increases binding affinity and/or specificity for CLCF1. In certain aspects, such a soluble CNTFR polypeptide includes a mutation (e.g., an amino acid substitution) at amino acid position 110, 174, 237, 287, or any combination thereof, relative to a CNTFR polypeptide having the amino acid sequence set forth in SEQ ID NO:8. An example mutation at position 110 is R110Q. An example mutation at position 174 is T174P. An example mutation at position 237 is S237F. Another example mutation at position 237 is S237Y. An example mutation at position 287 is I287F. In certain aspects, the soluble CNTFR polypeptide includes one or any combination (e.g., each) of the mutations R110Q, T174P, S237F/S237Y, and I287F.

In some embodiments, the soluble CNTFR polypeptide includes a mutation (e.g., an amino acid substitution) at amino acid position 110, 174, 177, 178, 237, 268, 269, 287, or any combination thereof, relative to a CNTFR polypeptide having the amino acid sequence set forth in SEQ ID NO:8.

In certain aspects, the soluble CNTFR polypeptide includes one or any combination (e.g., each) of the mutations R110Q, T174P, Y177H/Y177A, K178N/K178A, S237F/S237Y, T268A, D269A, and I287F.

A soluble CNTFR polypeptide according to one embodiment includes the amino acid sequence set forth in Table 5 below (SEQ ID NO:9). In Table 5, mutations are bold/underlined. In this example, the soluble CNTFR polypeptide includes a C-terminal truncation of amino acids 343-372 relative to a wild-type CNTF receptor having the amino acid sequence set forth in SEQ ID NO:8. In certain aspects, such a soluble CNTFR polypeptide does not include a signal peptide (underlined in Table 5).

TABLE 5
Amino Acid Sequence of an Example Soluble CNTFR Polypeptide

| | Amino Acid Sequence |
|---|---|
| Example Soluble CNTFR | MAAPVPWACCAVLAAAAAVVYAQRHSPQEAPHVQYERLGSDV |
| Polypeptide | TLPCGTANWDAAVTWRVNGTDLAPDLLNGSQLVLHGLELGHS |
| (SEQ ID NO: 9) | GLYACFHRDSWHLRHQVLLHVGLPPQEPVLSCRSNTYPKGFY |
| (R110Q, T174P, Y177H, | CSWHLPTPTYIPNTFNVTVLHGSKIMVCEKDPALKNRCHIRYMH |
| K178N, S237F, T268A, | LFSPIKHNVSISVSNALGHNATAITFDEFTIVKPDPPENVVARPV |
| D269A, I287F) | PSNPRRLEVTWQTPSTWPDPEFFPLKFFLRYRPLILDQWQHVE |
| | LSDGTAHTIAAAYAGKEYIIQVAAKDNEFGTWSDWSVAAHATP |
| | WTEEPRHLTTEAQAAETTTSTTSSLAPPPTTKICDPGELGS |

According to certain embodiments, a soluble CNTFR polypeptide of the present disclosure includes an amino acid sequence that has 70% or greater, 75% or greater, 80% or greater, 85% or greater, 90% or greater, 95% or greater, 99% or greater, or 100% identity to amino acids 23-342 of SEQ ID NO:8 or SEQ ID NO:9, or a fragment thereof, such as a fragment having a length of from 250 to 319 amino acids, 250 to 260 amino acids, 260 to 270 amino acids, 270 to 280 amino acids, 280 to 290 amino acids, 290 to 300 amino acids, 300 to 310 amino acids, or 310 to 319 amino acids. In addition to being soluble, such a CNTFR polypeptide may include one or more desirable features, such as reduced binding affinity for one or more ligand-CNTFR complex subunits (e.g., LIFR, gp130, or both), increased binding affinity/specificity for CLCF1, reduced binding affinity for a CNTFR ligand (e.g., CNTF, NP, etc.), and any combination thereof.

In some embodiments, a soluble CNTFR polypeptide includes one or more (e.g., each) of the amino acid substitutions R110Q, T174P, Y177H, K178N, S237F, T268A, D269A, and I287F, and an amino acid sequence that has 70% or greater, 75% or greater, 80% or greater, 85% or greater, 90% or greater, 95% or greater, 99% or greater, or 100% identity to amino acids 23-342 of SEQ ID NO:9.

According to some embodiments, a soluble CNTFR polypeptide of the present disclosure is fused to an Fc domain. Such fusion proteins are described in greater detail below. The amino acid sequence of an example soluble CNTFR polypeptide fused to an Fc domain is set forth in Table 6 below (with the Fc domain underlined and the signal peptide italicized).

TABLE 6
Amino Acid Sequence of an Example Soluble CNTFR Polypeptide-Fc Fusion

| | Amino Acid Sequence |
|---|---|
| Example Soluble | MAAPVPWACCAVLAAAAAVVYAQRHSPQEAPHVQYERLGSDVTLP |
| CNTFR | CGTANWDAAVTWRVNGTDLAPDLLNGSQLVLHGLELGHSGLYACF |
| Polypeptide-Fc | HRDSWHLRHQVLLHVGLPPQEPVLSCRSNTYPKGFYCSWHLPTPT |
| Fusion (SEQ ID | YIPNTFNVTVLHGSKIMVCEKDPALKNRCHIRYMHLFSPIKHNVSISV |
| NO: 10) | SNALGHNATAITFDEFTIVKPDPPENVVARPVPSNPRRLEVTWQTPS |
| (R110Q, T174P, | TWPDPEFFPLKFFLRYRPLILDQWQHVELSDGTAHTIAAAYAGKEYII |
| Y177H, K178N, | QVAAKDNEFGTWSDWSVAAHATPWTEEPRHLTTEAQAAETTTSTT |
| S237F, T268A, | SSLAPPPTTKICDPGELGSRRLEPRGPTIKPCPPCKCPAPNLLGGPS |
| D269A, I287F) | VFIFPPKIKDVLMISLSPIVTCVVVDVSEDDPDVQISWFVNNVEVHTA |
| | QTQTHREDYNSTLRVVSALPIQHQDWMSGKEFKCKVNNKDLPAPIE |
| | RTISKPKGSVRAPQVYVLPPPEEEMTKKQVTLTCMVTDFMPEDIYVE |
| | WTNNGKTELNYKNTEPVLDSDGSYFMYSKLRVEKKNWVERNSYSC |
| | SVVHEGLHNHHTTKSFSRTPGK |

According to certain embodiments, a soluble CNTFR polypeptide-Fc fusion includes an amino acid sequence that has 70% or greater, 75% or greater, 80% or greater, 85% or greater, 90% or greater, 95% or greater, 99% or greater, or 100% identity to amino acids 23-578 of SEQ ID NO:10, or a fragment thereof, such as a fragment having a length of from 450 to 555 amino acids, 500 to 555 amino acids, 525 to 555 amino acids, 540 to 555 amino acids, or 550 to 555 amino acids. In certain aspects, such a soluble CNTFR polypeptide-Fc fusion does not include a signal peptide (italicized in Table 6).

According to certain embodiments, a soluble CNTFR polypeptide-Fc fusion includes one or more (e.g., each) of the amino acid substitutions R110Q, T174P, Y177H, K178N, S237F, T268A, D269A, and I287F, and an amino acid sequence that has 70% or greater, 75% or greater, 80% or greater, 85% or greater, 90% or greater, 95% or greater, 99% or greater, or 100% identity to amino acids 23-578 of SEQ ID NO:10, or a fragment thereof, such as a fragment having a length of from 450 to 555 amino acids, 500 to 555 amino acids, 525 to 555 amino acids, 540 to 555 amino acids, or 550 to 555 amino acids. In certain aspects, such a soluble CNTFR polypeptide-Fc fusion does not include a signal peptide (italicized in Table 6).

Fusion Proteins and Conjugates

In certain aspects, the agent administered to the individual (e.g., any of the agents described elsewhere herein) is stably associated with (e.g., fused to, conjugated to, or otherwise attached to) a heterologous moiety.

In some embodiments, provided are fusion proteins in which the agent is a polypeptide fused to a heterologous polypeptide. Heterologous polypeptides of interest include, but are not limited to, an Fc domain (e.g., a human or mouse Fc domain), an albumin, a transferrin, XTEN, a homo-amino acid polymer, a proline-alanine-serine polymer, an elastin-like peptide, or any combination thereof. In certain aspects, the heterologous polypeptide increases the stability and/or serum half-life of the polypeptide agent upon its administration to the individual, as compared to the same polypeptide agent which is not fused to the heterologous polypeptide. In certain aspects, provided are fusion proteins that include any of the polypeptide agents fused to a human Fc domain (e.g., a full-length human Fc domain or fragment thereof). A non-limiting example of a human Fc domain that may be fused to any of the polypeptide agents described elsewhere herein is a human IgG1 Fc domain having the sequence set forth in Table 7 below (SEQ ID NO:11), or a fragment thereof.

TABLE 7
Amino Acid Sequence of an Example Human Fc Domain

| | Amino Acid Sequence |
|---|---|
| Example Human | DKTHTCPPCPAPELLGGPSVFLFPPKPKDTLMISRTPEVTCVVVDVSHE |
| Fc Domain | DPEVKFNWYVDGVEVHNAKTKPREEQYNSTYRVVSVLTVLHQDWLNG |
| (SEQ ID NO: 11) | KEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDK |
| | SRWQQGNVFSCSVMHEALHNHYTQKSLSLSPGK |

According to certain embodiments, provided are conjugates in which the agent is conjugated to a moiety. Moieties of interest include, but are not limited to, polyethylene glycol (PEG), an anti-cancer drug, a detectable label, and combinations thereof.

Anti-cancer drugs of interest include those that inhibit cell proliferation and/or kill cancer cells. Such may vary and include cytostatic agents and cytotoxic agents (e.g., an agent capable of killing a target cell tissue with or without being internalized into a target cell). In certain aspects, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof. According to certain embodiments, the agent is a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

Detectable labels include labels that may be detected in an application of interest (e.g., in vitro and/or in vivo research and/or clinical applications). Detectable labels of interest include radioisotopes, enzymes that generate a detectable product (e.g., horseradish peroxidase, alkaline phosphatase, etc.), fluorescent proteins, paramagnetic atoms, and the like. In certain aspects, the CNTFR ligand is conjugated to a specific binding partner of detectable label (e.g., conjugated to biotin such that detection may occur via a detectable label that includes avidin/streptavidin).

According to certain embodiments, the agent is a labeling agent that finds use in in vivo imaging, such as near-infrared (NIR) optical imaging, single-photon emission computed tomography (SPECT)/CT imaging, positron emission tomography (PET), nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain aspects, the labeling agent is an in vivo imaging agent that finds use in near-infrared (NIR) imaging applications, which agent is selected from a Kodak X-SIGHT dye, Pz 247, DyLight 750 and 800 Fluors, Cy 5.5 and 7 Fluors, Alexa Fluor 680 and 750 Dyes, IRDye 680 and 800CW Fluors. According to certain embodiments, the labeling agent is an in vivo imaging agent that finds use in SPECT imaging applications, which agent is selected from ⁹⁹mTc, ¹¹¹In, ¹²³In, ²⁰¹Tl, and ¹³³Xe. In certain aspects, the labeling agent is an in vivo imaging agent that finds use in positron emission tomography (PET) imaging applications, which agent is selected from ¹¹C, ¹³N, ¹⁵O, ¹⁸F, ⁶⁴Cu, ⁶²Cu, ¹²⁴I, ⁷⁶Br, ⁸²Rb and ⁶⁸Ga.

Linkers that find use in the conjugates of the present disclosure include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof.

Numerous strategies are available for linking the agent to a moiety of interest through a linker. For example, the moiety of interest may be derivatized by covalently attaching the linker to the moiety, where the linker has a functional group capable of reacting with a “chemical handle” on the agent. The functional group on the linker may vary and may be selected based on compatibility with the chemical handle on the agent. According to one embodiment, the chemical handle on the agent is provided by incorporation of an unnatural amino acid having the chemical handle into the agent. Such an unnatural amino acid may be incorporated into the agent, e.g., via chemical synthesis or recombinant approaches, e.g., using a suitable orthogonal amino acyl tRNA synthetase-tRNA pair for incorporation of the unnatural amino acid during translation in a host cell.

The functional group of an unnatural amino acid present in the agent may be an azide, alkyne, alkene, amino-oxy, hydrazine, aldehyde, nitrone, nitrile oxide, cyclopropene, norbornene, iso-cyanide, aryl halide, boronic acid, or other suitable functional group, and the functional group on the linker is selected to react with the functional group of the unnatural amino acid (or vice versa).

Administration

As summarized above, the methods of the present disclosure include methods of treating a KRAS mutant cancer in an individual. By “treating” or “treatment” is meant at least an amelioration of the symptoms associated with the KRAS mutant cancer of the individual, where amelioration is used in a broad sense to refer to at least a reduction in the magnitude of a parameter, e.g. symptom, associated with the KRAS mutant cancer being treated. As such, treatment also includes situations where the KRAS mutant cancer, or at least symptoms associated therewith, are completely inhibited, e.g., prevented from happening, or stopped, e.g., terminated, such that the individual no longer suffers from the KRAS mutant cancer, or at least the symptoms that characterize the KRAS mutant cancer.

The agent that inhibits CLCF1-CNTFR signaling is administered to the individual in a therapeutically effective amount. In some embodiments, a therapeutically effective amount of the agent (e.g., present in pharmaceutical composition including same) is an amount that, when administered alone (e.g., in monotherapy) or in combination (e.g., in combination therapy) with one or more additional therapeutic agents, in one or more doses, is effective to reduce the symptoms of the KRAS mutant cancer in the individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the agent. According to some embodiments, the methods of the present disclosure inhibit growth, metastasis and/or invasiveness of cancer cells of the KRAS mutant cancer when the agent is administered in an effective amount.

Dosing is dependent on severity and responsiveness of the KRAS mutant cancer to be treated. Optimal dosing schedules can be calculated from measurements of drug accumulation in the body of the individual. The administering physician can determine optimum dosages, dosing methodologies and repetition rates. Optimum dosages may vary depending on the relative potency of individual agent and can generally be estimated based on EC₅₀s found to be effective in in vitro and in vivo animal models, etc. In general, dosage is from 0.01 μg to 100 g per kg of body weight, and may be given once or more daily, weekly, monthly or yearly. The treating physician can estimate repetition rates for dosing based on measured residence times and concentrations of the agent in bodily fluids or tissues. Following successful treatment, it may be desirable to have the individual undergo maintenance therapy to prevent the recurrence of the disease state, where the agent is administered in maintenance doses, ranging from 0.01 μg to 100 g per kg of body weight, once or more daily, to once every several months, once every six months, once every year, or at any other suitable frequency.

The therapeutic methods of the present disclosure may include administering a single type of agent that inhibits CLCF1-CNTFR signaling to the individual, or may include administering two or more types of agents that inhibit CLCF1-CNTFR signaling by administration of a cocktail of different agents that inhibit CLCF1-CNTFR signaling, e.g., a first agent that specifically binds CNTFR and inhibits signaling through CNTFR (e.g., any of the engineered ligands described herein) and a second agent that specifically binds CLCF1 and inhibits signaling through CNTFR, e.g., any of the engineered soluble CNTFR polypeptides described herein.

The agent may be administered to an individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, oral, and other enteral and parenteral routes of administration. Routes of administration may be combined, if desired, or adjusted depending upon the particular agent and/or the desired effect. The agent may be administered in a single dose or in multiple doses. In some embodiments, the agent is administered parenterally, e.g., intravenously, intraarterially, or the like. In some embodiments, the agent is administered by injection, e.g., for systemic delivery (e.g., intravenous infusion) or to a local site, e.g., intratumoral injection.

The agent can be incorporated into a variety of formulations for administration to the individual. More particularly, the agent can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, emulsions, injections, inhalants and aerosols.

Formulations of the agent suitable for administration to an individual (e.g., suitable for human administration) are generally sterile and may further be free of detectable pyrogens or other contaminants contraindicated for administration to a patient according to a selected route of administration.

In pharmaceutical dosage forms, the agent can be administered alone or in appropriate association, as well as in combination, with a second pharmaceutically active compound, e.g., a second anti-cancer agent (including but not limited to small molecule anti-cancer agents). The following methods and carriers/excipients are merely examples and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent can be formulated for parenteral (e.g., intravenous, intratumoral, intra-arterial, intraosseous, intramuscular, intracerebral, intracerebroventricular, intrathecal, subcutaneous, etc.) administration. In certain aspects, the agent is formulated for injection by dissolving, suspending or emulsifying the agent in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent may be prepared by mixing the agent having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the agent against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the agent, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

As summarized above, the present disclosure provides kits. In certain embodiments, the kits find use in practicing the methods of the present disclosure. According to some embodiments, a kit of the present disclosure includes an agent that inhibits CLCF1-CNTFR signaling, and instructions for administering the agent to an individual identified as having a KRAS mutant cancer.

A kit of the present disclosure may include any of the agents that inhibit CLCF1-CNTFR signaling described in the Methods section above, which description is incorporated but not reiterated herein for purposes of brevity. By way of example, a subject kit may include any of the engineered ligands, soluble CNTFR polypeptides, etc. described in the Methods section above.

In certain embodiments, the instructions of a kit of the present disclosure includes instructions for administering the agent to an individual identified as having a KRAS mutant lung cancer. For example, the instructions may include instructions for administering the agent to an individual identified as having a KRAS mutant non-small cell lung cancer (NSCLC). When a kit includes instructions for administering the agent to an individual identified as having a KRAS mutant NSCLC, the instructions may include instructions for administering the agent to an individual identified as having a KRAS mutant lung adenocarcinoma (LUAD).

According to some embodiments, a kit of the present disclosure includes instructions for administering the agent to an individual identified as having a KRAS mutant cancer comprising an amino acid substitution at position 12 of human KRAS, and wherein numbering is as in SEQ ID NO:1. In certain embodiments, such a kit may include instructions for administering the agent to an individual identified as having a KRAS mutant cancer comprising an amino acid substitution selected from the group consisting of: G12A, G12C, G12D, G12S , and G12V.

The subject kits may include a quantity of the agent that inhibits CLCF1-CNTFR signaling (e.g., present in a pharmaceutical composition comprising the agent and a pharmaceutically acceptable carrier), present in unit dosages, e.g., ampoules, or a multi-dosage format. As such, in certain embodiments, the kits may include one or more (e.g., two or more) unit dosages (e.g., ampoules) of a composition that includes the agent. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular agent employed, the effect to be achieved, and the pharmacodynamics associated with the agent, in the individual. In yet other embodiments, the kits may include a single multi dosage amount of the composition.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Expression of CLCF1 and CNTFR and the Oncogenic Effect of the CLCF1-CNTFR Signaling in Human LUAD

Analysis of public gene expression data indicated that CLCF1 is significantly upregulated in lung adenocarcinoma (LUAD) compared to normal lung (data not shown). High expression of CLCF1 was associated with decreased survival in patients with KRAS mutation [Cox hazard ratio: 2.53 (95% CI 1.43-4.48); p-value: 0.001] but not in patients without KRAS mutation [Cox hazard ratio: 0.86 (95% CI 0.51-1.4); p-value: 0.56]. This result suggested a specific role of CLCF1 signaling in KRAS driven oncogenesis. CNTFR expression in KRAS mutant LUAD did not show the same pattern [Cox hazard ratio: 1.36 (95% CI 0.65-2.82); p-value: 0.41]. Prior work indicated that in mouse lung tumors, cancer-associated fibroblasts (CAFs) are the primary source of CLCF1. To determine whether human lung CAFs also provide a source of CLCF1, CAFs were isolated from human lung cancer patients and matched normal lung fibroblasts (NLFs). Expression of CLCF1 was significantly elevated in six of eight human CAFs compared to patient-matched NLFs. However, the LUAD cell lines tested also secrete CLCF1, suggesting the existence of both paracrine and autocrine signaling for this cytokine in human LUAD.

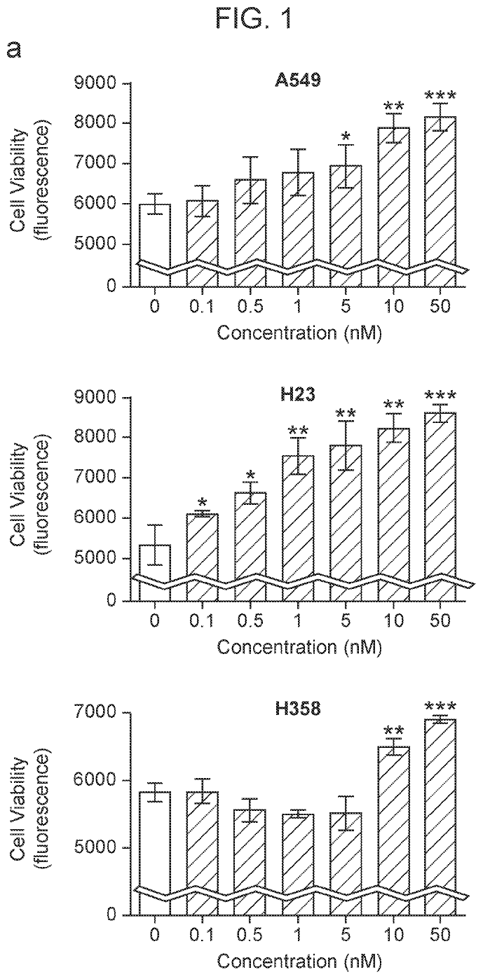

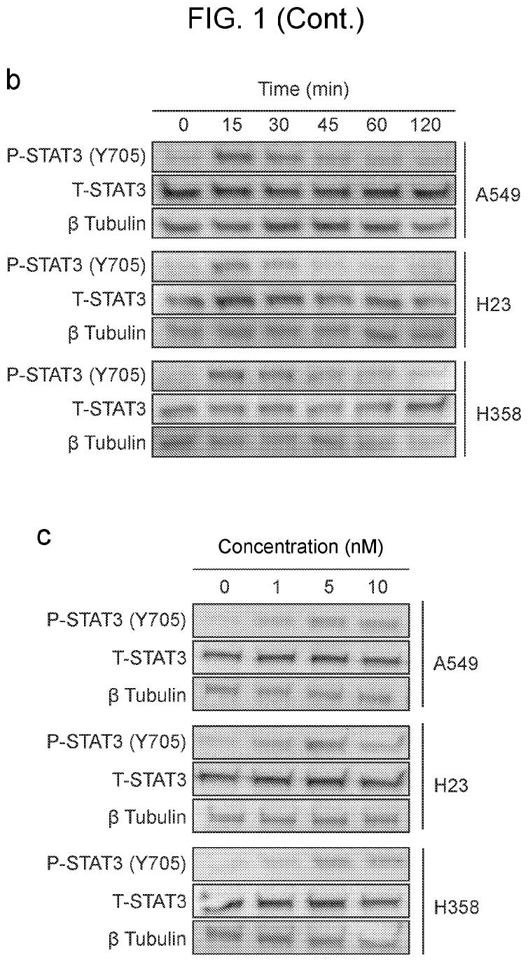

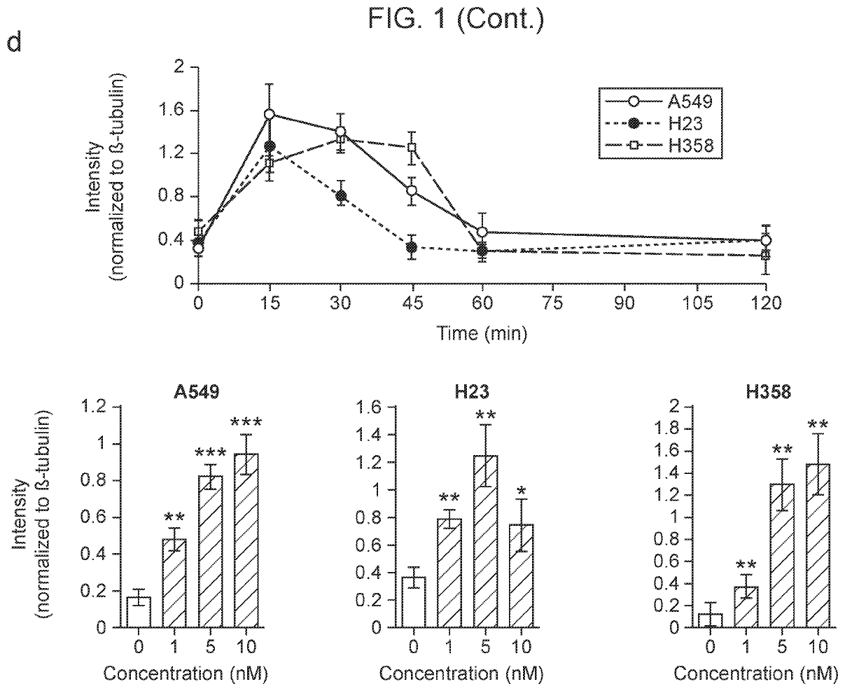

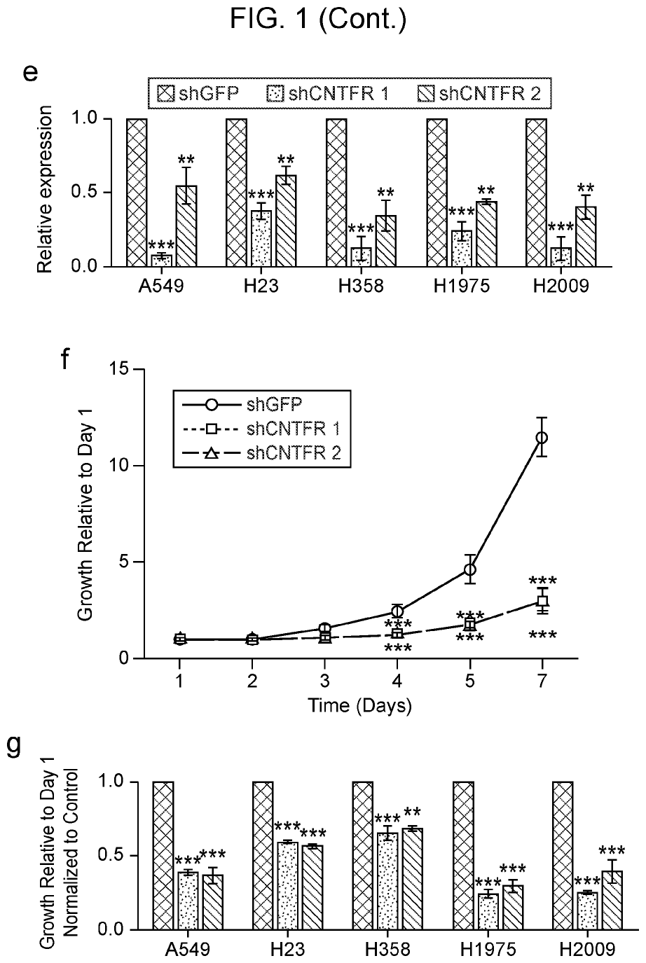

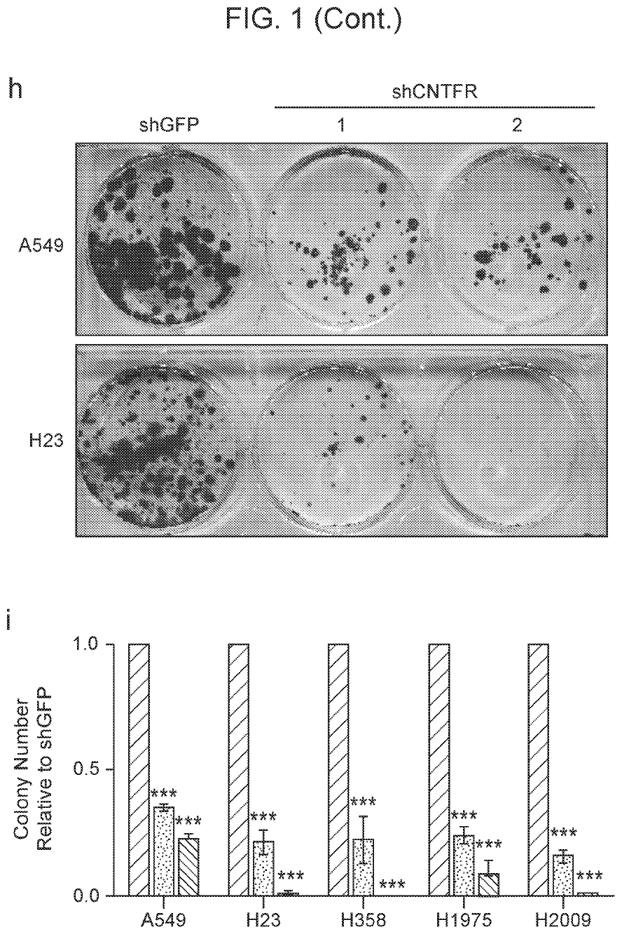

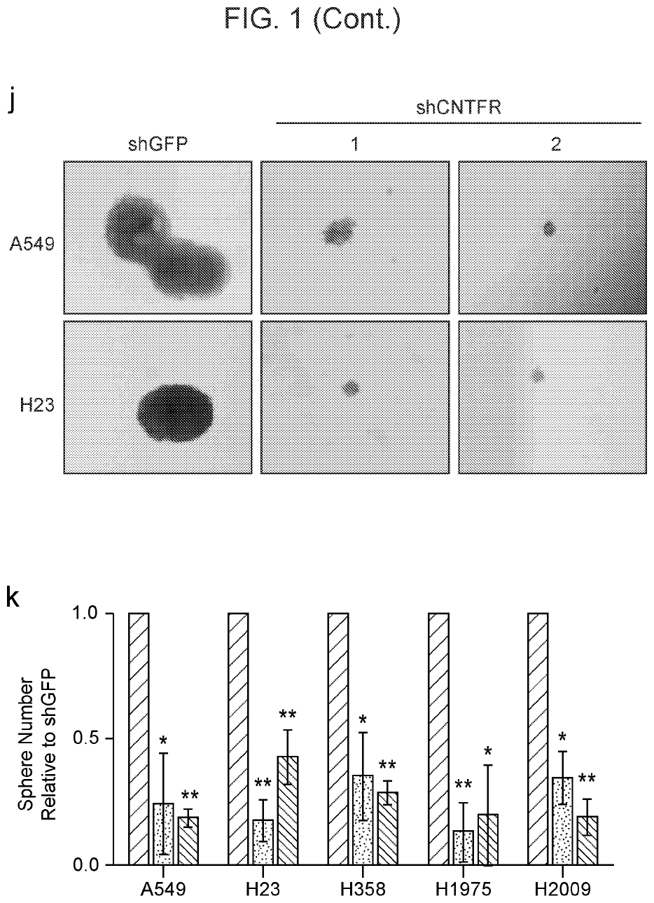

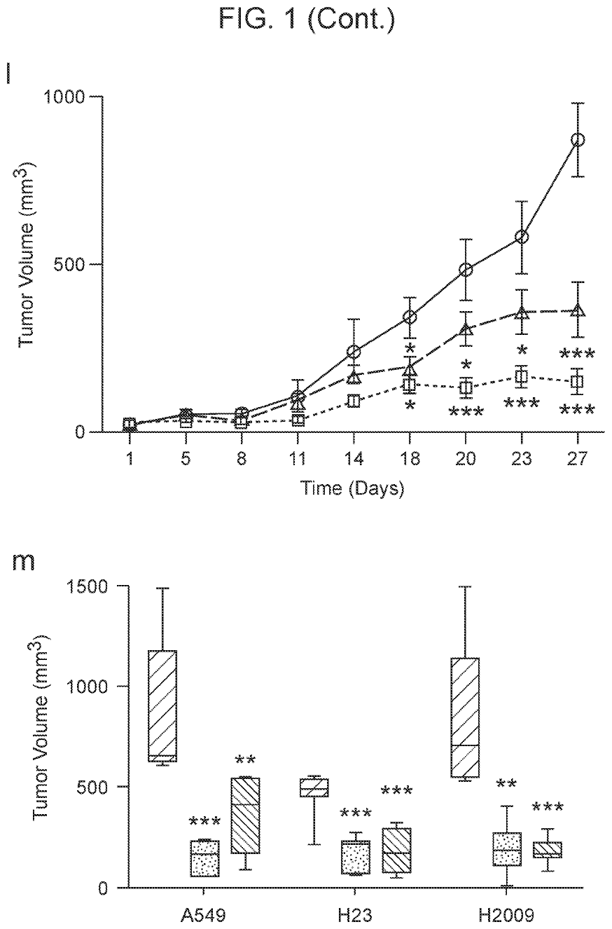

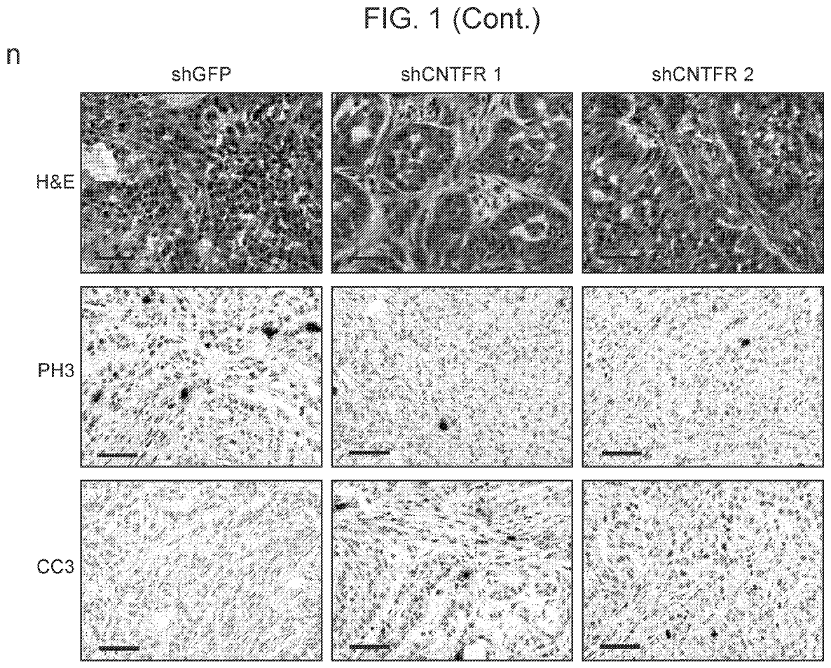

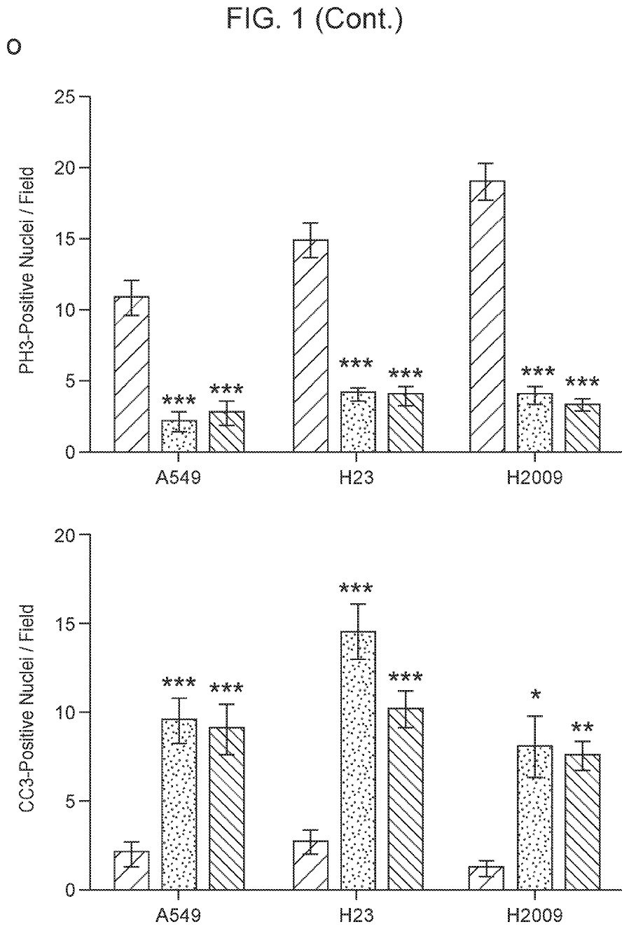

Next, the functional role of CLCF1 in cell lines was evaluated. Exposure to recombinant CLCF1 increased proliferation in all LUAD cell lines examined (FIG. 1, panel A). Ligand binding to the CNTFR/LIFR/gp130 complex leads to phosphorylation of gp130 and activation of downstream signals including STAT3 and ERK. Thus, as expected, CLCF1 induced phosphorylation of STAT3 (FIG. 1, panels B-D). To further probe the functional significance of CLCF1-CNTFR signaling in human lung cancer, RNA interference was used to decrease the amount of CNTFR at the cell surface. Knock-down using two different shRNAs significantly decreased viability of all five LUAD cell lines tested (FIG. 1, panels E-G). CNTFR knock-down also suppressed clonogenic growth of LUAD cell lines (FIG. 1, panels H and I) and led to decreased size and number of spheres in 3D culture (FIG. 1, panels J and K). Evaluated next was whether CNTFR knock-down would influence tumor growth in vivo. CNTFR knock-down in all three LUAD cell lines tested decreased xenograft formation (FIG. 1, panels L and M). Moreover, tumors formed from LUAD cells with CNTFR knock-down exhibited a lower proliferative index and higher levels of apoptosis compared to control tumors (FIG. 1, panels N and O). To test whether the contribution is mostly paracrine or autocrine, CLCF1 was knocked down in H2009 and the cells implanted as xenografts. An equally efficacious decrease in tumor growth was observed in both the CLCF1 and the CNTFR knockdown tumors, suggesting that at least in subcutaneous xenograft models the source of CLFC1 is primarily autocrine secretion from the tumor cells themselves.

To determine the mechanism of action of CNTFR blockade in LUAD, evaluated was the effect of knock-down on the MAPK, AKT, and STAT3 signaling pathways, all previously identified as activated downstream of gp130. Phosphorylation of ERK and S6 were decreased in tumors after CNTFR knock-down, indicating effects on the MAPK/ERK and AKT pathways, respectively. Decreased phosphorylation of STAT3 was also observed. Taken together, these results indicate that CLCF1-CNTFR signaling is active in LUAD, has a pro-oncogenic role, and the mechanism of CNTFR inhibition involves dampening of the activity of several signaling cascades including STAT3, ERK, and AKT signaling.

### Example 2—Engineering a Soluble Receptor Decoy to Inhibit the CLCF1-CNTFR Signaling Axis

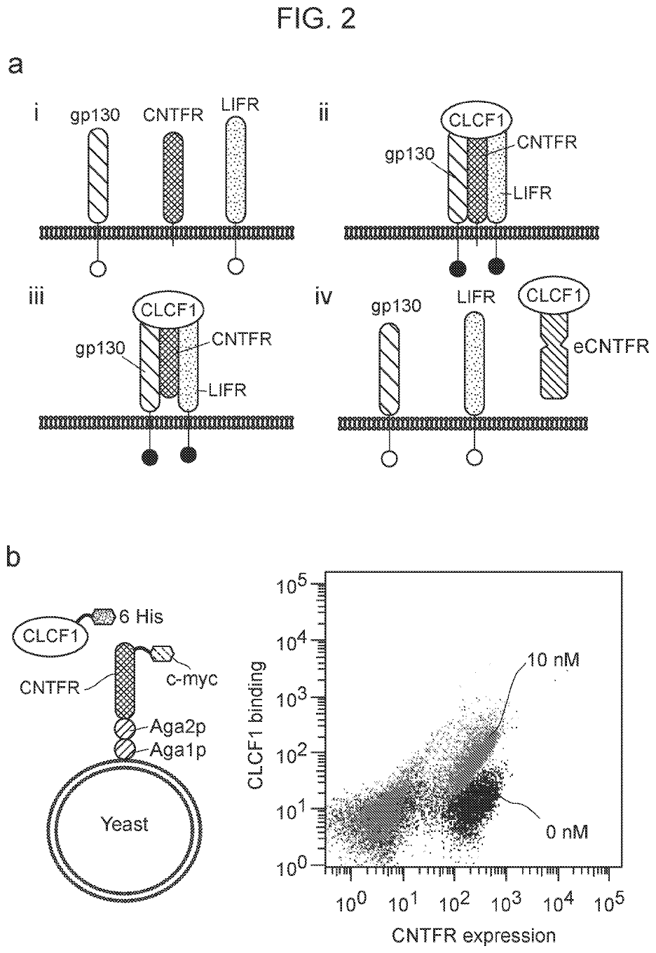

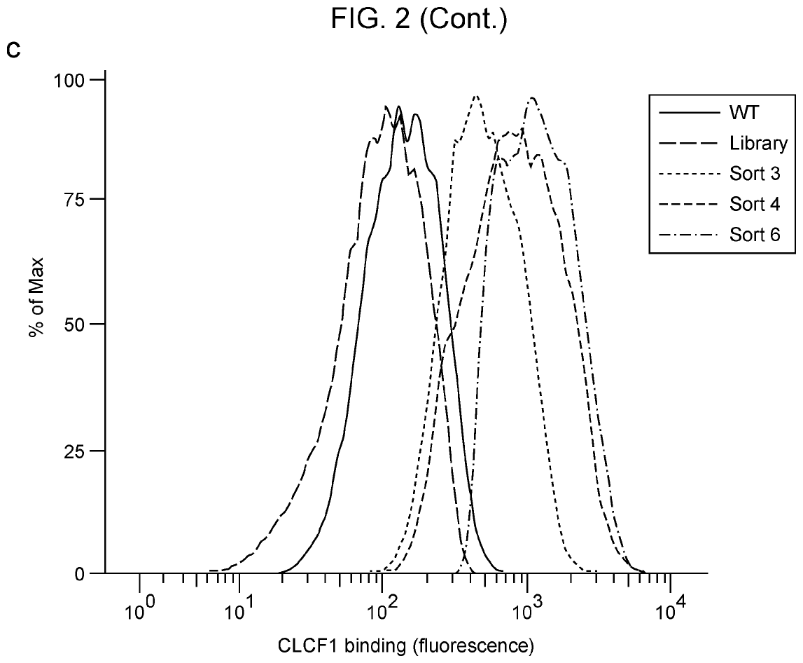

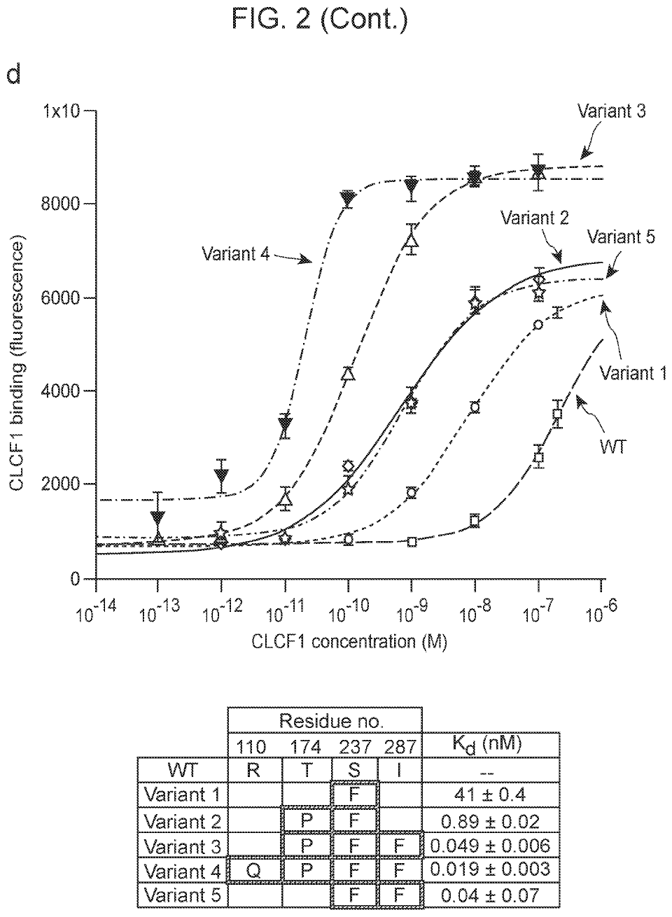

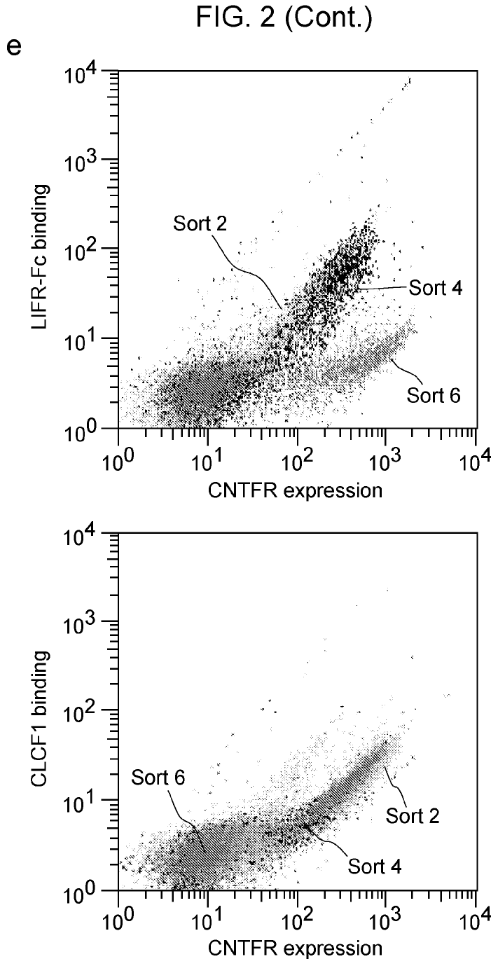

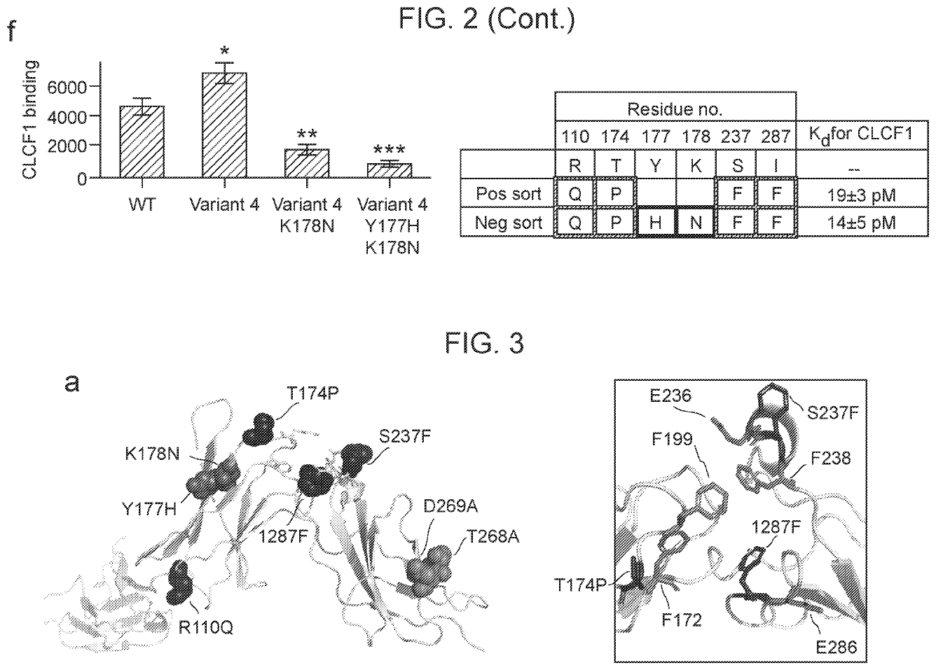

The functional studies above support that inhibition of CLCF1-CNTFR signaling could be a therapeutic opportunity in lung cancer. Therefore, we sought to identify an effective strategy to target this pathway. CNTFR is anchored to the cell surface via a glycosylphosphatidylinositol (GPI) linkage that forms following proteolytic cleavage of a C-terminal propeptide (FIG. 2, panel A, i). When bound to CLCF1, CNTFR forms a complex with gp130 and LIFR (FIG. 2, panel A, ii). Without the propeptide, CNTFR is secreted from the membrane but can still bind to CLCF1 and activate downstream signaling, even in cells that do not express CNTFR (FIG. 2, panel A, iii). Thus, effective blockade of CLCF1 requires both increasing binding of the decoy to CLCF1 and decreasing binding to gp130 and LIFR (FIG. 2, panel A, iv).

Directed evolution was used to engineer a soluble CNTFR variant with stronger affinity for CLCF1 and lack of binding to gp130 and LIFR. It was hypothesized that such a molecule would act as an efficient ligand trap and antagonize CLCF1-mediated oncogenic signaling. To develop a high-affinity receptor decoy, DNA encoding the extracellular domain of CNTFR was subjected to random mutagenesis via error-prone PCR. The corresponding protein library (˜10⁸ transformants) was displayed as fusions on the yeast cell surface (FIG. 2, panel B). The library was screened to enrich for variants with increased CLCF1 binding using flow cytometry. After 3 rounds of screening, T174P and S237F appeared as consensus mutations, with substantial diversity observed at other amino acid positions. To probe additive effects of these mutations, 20 randomly selected distinct clones from the sorted populations were shuffled using the Staggered Extension Process (StEP) method to create a second library. A combination of equilibrium binding and kinetic off-rate screens were used to sort this library to impose increased screening stringency (FIG. 2, panel C). After three rounds of screening, combinations of four consensus mutations (R110Q, T174P, S237F, and I287F) emerged. Quantitative yeast-displayed binding studies indicated that each of these mutations contributed to the higher binding affinity for CLCF1 (FIG. 2, panel D), with the combination of all four mutations leading to an apparent K_{d} of 20 pM. This CNTFR variant (variant 4) was carried forward for further optimization.

Since CLCF1-CNTFR binding activates downstream signaling through heterodimerization of LIFR and gp130, modifying CNTFR to reduce or prevent formation of this complex while sequestering CLCF1 is beneficial for inhibitory activity. It was confirmed that yeast-displayed CNTFR does complex with gp130 and LIFR in a CLCF1-dependent manner. Therefore, CNTFR variant 4 was further engineered to decrease its binding to the co-receptors. Random mutations were introduced into CNTFR variant 4 using error-prone PCR, and the resulting library was incubated with CLCF1 and screened for variants with decreased binding signal for LIFR by flow cytometry (FIG. 2, panel E). Two consensus mutations (Y177H and K178N) were identified that reduced binding to LIFR (FIG. 2, panel F). A final variant, eCNTFR, was created that combines these two mutations, the four mutations that confer high affinity CLCF1 binding, and an additional two alanine substitutions (T268A and D269A) shown to weaken binding to gp130.

### Example 3—Characterization of Soluble eCNTFR

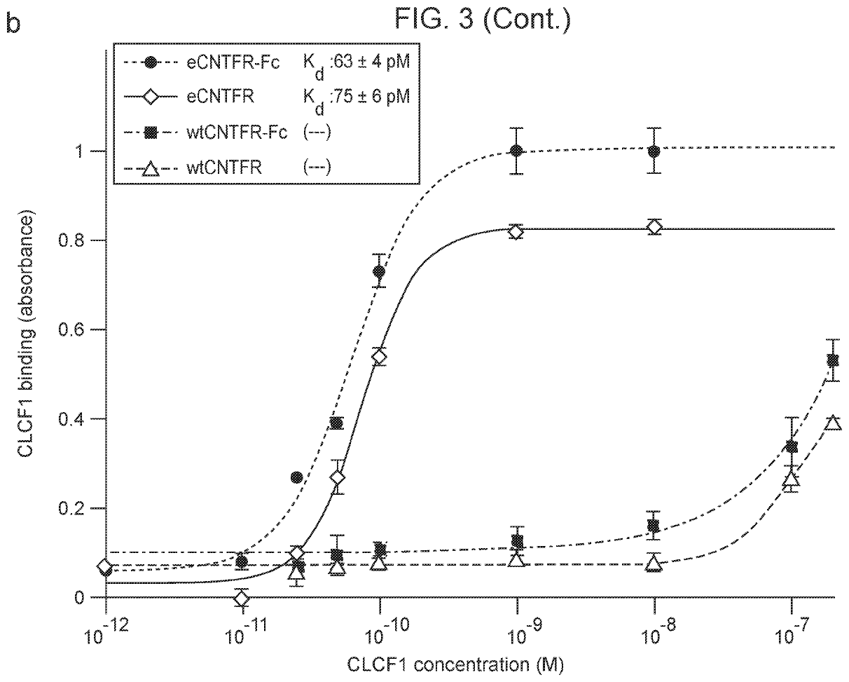

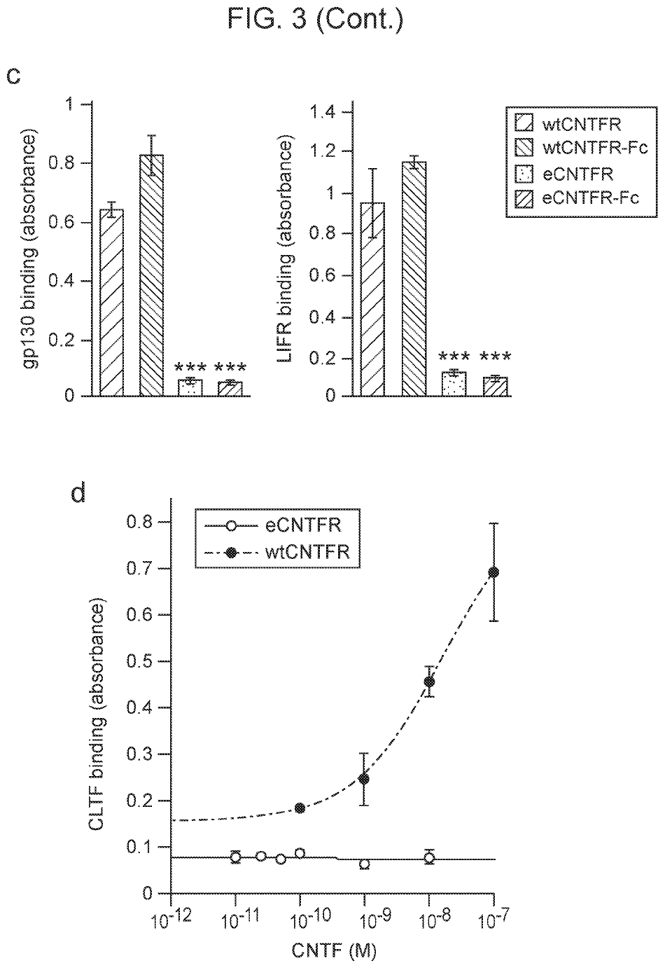

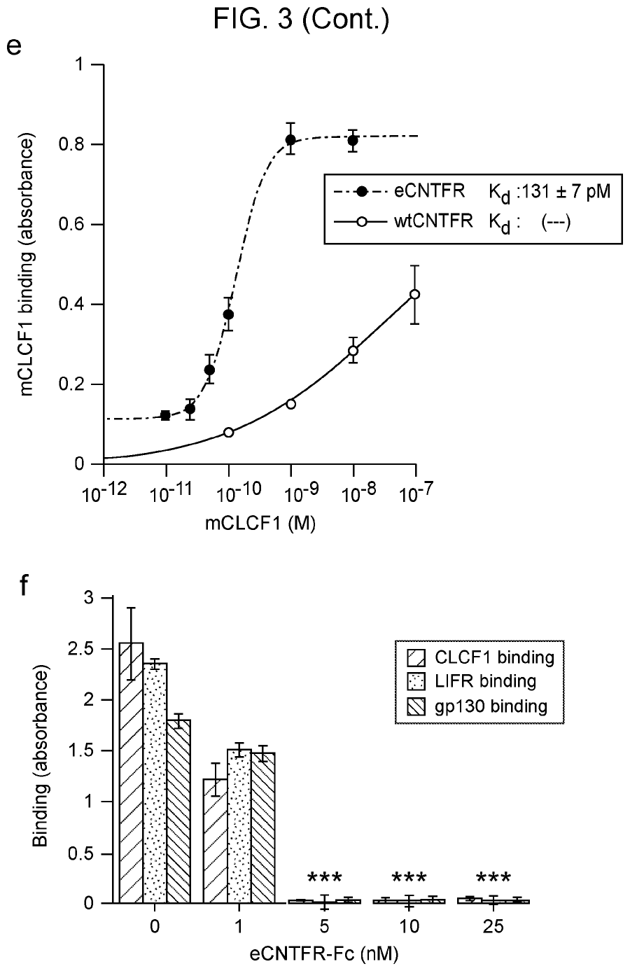

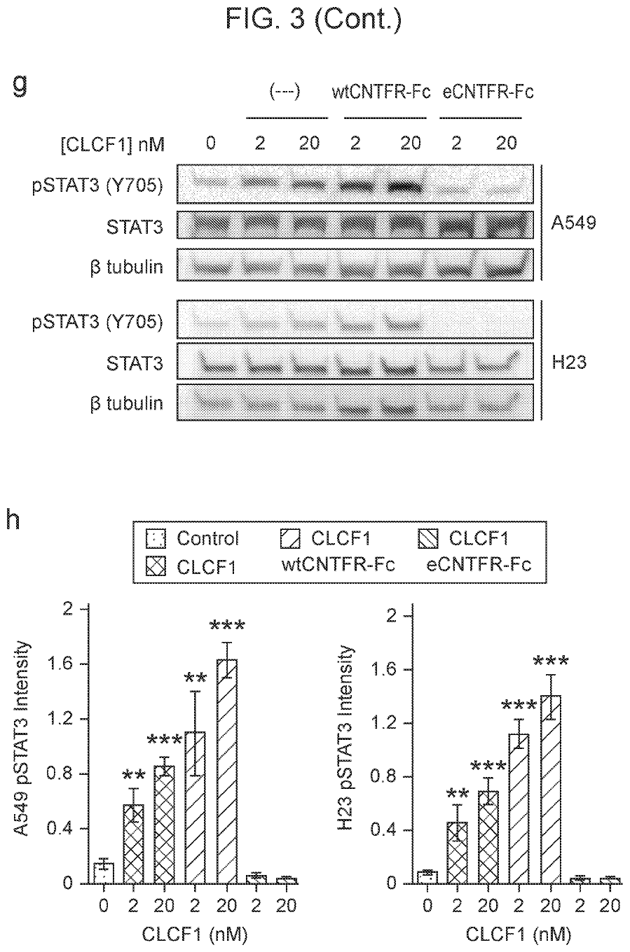

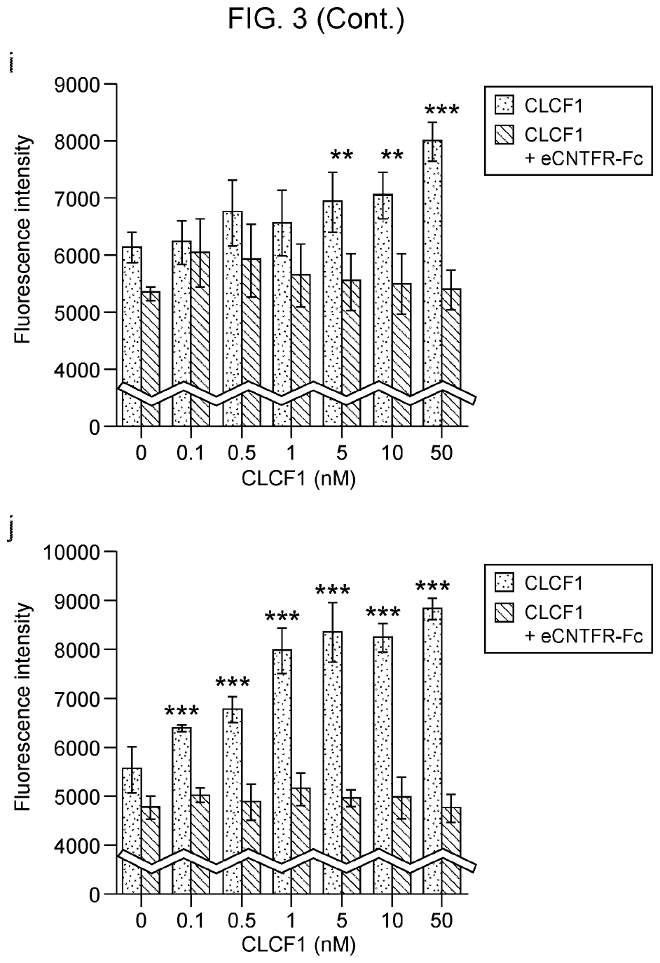

As structural information on full-length CNTFR is unavailable, wtCNTFR and eCNTFR were modeled using the Phyre 2 server to predict the three-dimensional locations of mutations in eCNTFR. Three of the four mutations identified by affinity maturation (T174P, S237F, and S287F) were proximal to the aromatic cluster (F172, F199, and F238) and the conserved residues (E236 and E286) that have been shown to be important for cytokine binding (FIG. 3, panel A). Soluble eCNTFR was recombinantly expressed with a C-terminal hexahistidine tag (eCNTFR-His) or as an N-terminal fusion to an antibody Fc domain (eCNTFR-Fc) and affinity to CLCF1 was measured using a microtiter plate-based assay. Both eCNTFR-His and eCNTFR-Fc exhibited picomolar binding affinity to CLCF1 (FIG. 3, panel B). In comparison, CLCF1 binding affinity was too weak to be quantified for soluble wild-type CNTFR constructs (wtCNTFR-His and wtCNTFR-Fc). A similar approach was used to characterize binding interactions with gp130 and LIFR. In these experiments, eCNTFR constructs showed no detectable binding to gp130 and LIFR, in contrast to wtCNTFR constructs, which bound to both receptors (FIG. 3, panel C). Increasing the size of a protein to avoid glomerular filtration can significantly increase serum half-life, and the Fc domain can further increase half-life through FcRn-mediated recycling. Therefore, the eCNTFR-Fc fusion was used to further evaluate the effect of eCNTFR in animal models of LUAD.

CNTF is another ligand for CNTFR, and CNTF-mediated signaling is important for neuronal cell survival. Engineering binding selectivity of eCNTFR-Fc to CLCF1 over CNTF could help minimize any potential side effects from inhibiting CNTF signaling. Additionally, while CLCF1 is known to act only through CNTFR, CNTF also binds to the IL-6 receptor (IL-6R), suggesting that CLCF1 and CNTF have unique functional roles in regulating signaling pathways. wtCNTFR-Fc exhibited binding to recombinantly produced CNTF, while eCNTFR-Fc did not (FIG. 3, panel D). These results are consistent with yeast-displayed binding data for wtCNTFR and eCNTFR and indicate that affinity maturation of CNTFR for CLCF1 led to increased specificity towards CLCF1 and decreased binding to CNTF. In addition, eCNTFR-Fc bound to mouse CLCF1 with high affinity as compared to wtCNTFR-Fc, indicating its utility for in vivo experiments in which CLCF1 is of mouse origin (FIG. 3, panel E). Importantly, mouse CLCF1 can activate CNTFR in human cells.

To assess whether eCNTFR-Fc could effectively sequester CLCF1 and block receptor complex formation, a competition binding assay was designed to measure the effect of eCNTFR-Fc on the interaction between wtCNTFR and each of the other subunits of the receptor complex. Incubating eCNTFR-Fc in wtCNTFR-His-coated wells prevented CLCF1, LIFR, and gp130 constructs from interacting with wtCNTFR-His (FIG. 3, panel F). To determine whether eCNTFR-Fc could effectively neutralize CLCF1 and inhibit gp130 signaling, LUAD cells were stimulated with CLCF1 in the presence and absence of the soluble CNTFR constructs. While wtCNTFR-Fc increased phosphorylation of STAT3 (Tyr705), eCNTFR-Fc decreased phosphorylation in both cell lines tested (FIG. 3, panels G and H). Furthermore, incubation with eCNTFR-Fc inhibited CLCF1-mediated viability (FIG. 3, panels I and J).

### Example 4—eCNTFR-Fc Selectively Inhibits KRAS Mutant Cells by Decreasing Ras-GTP Loading

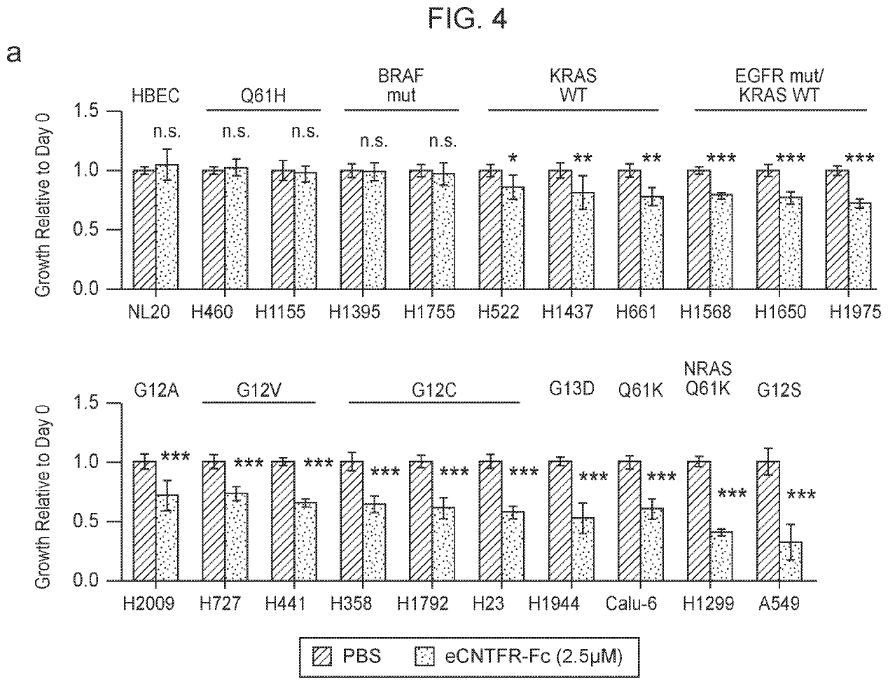

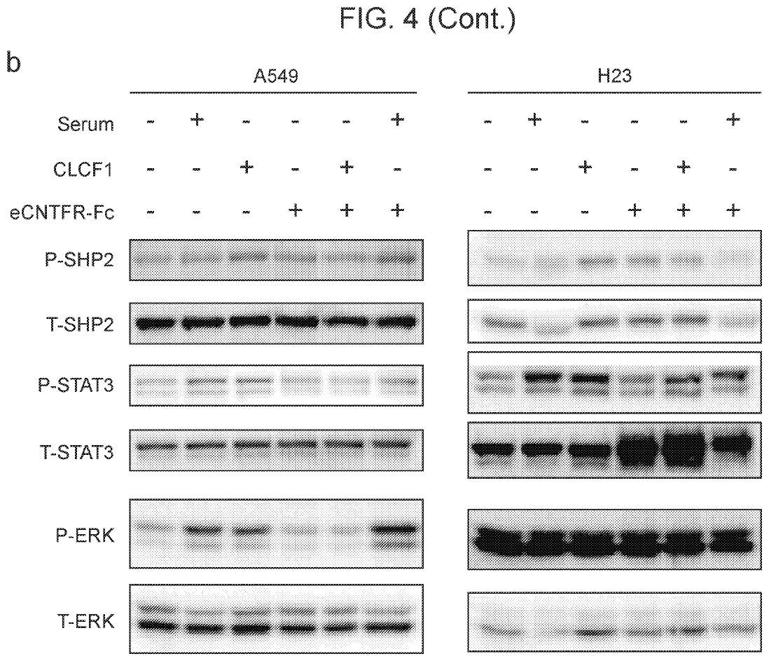

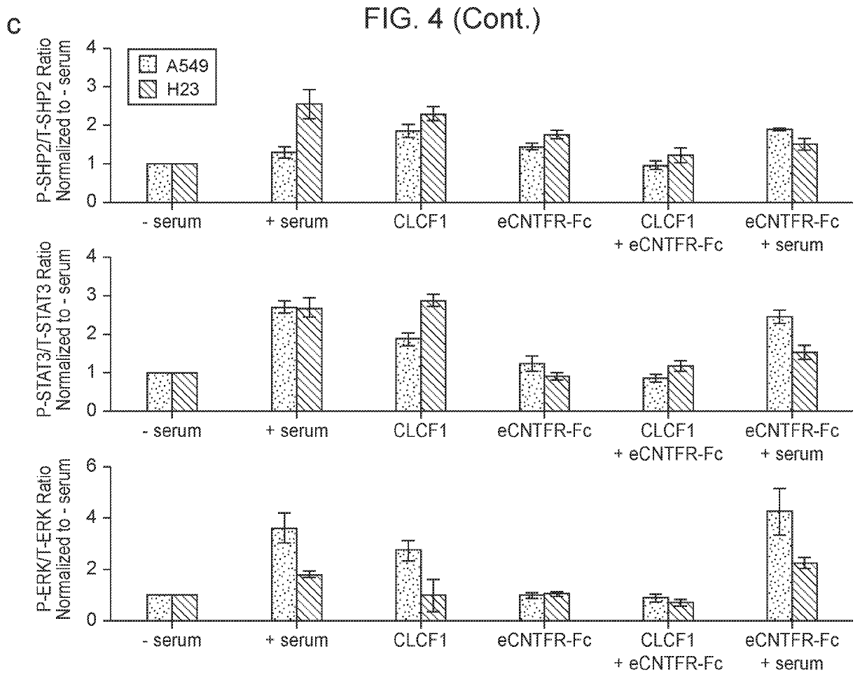

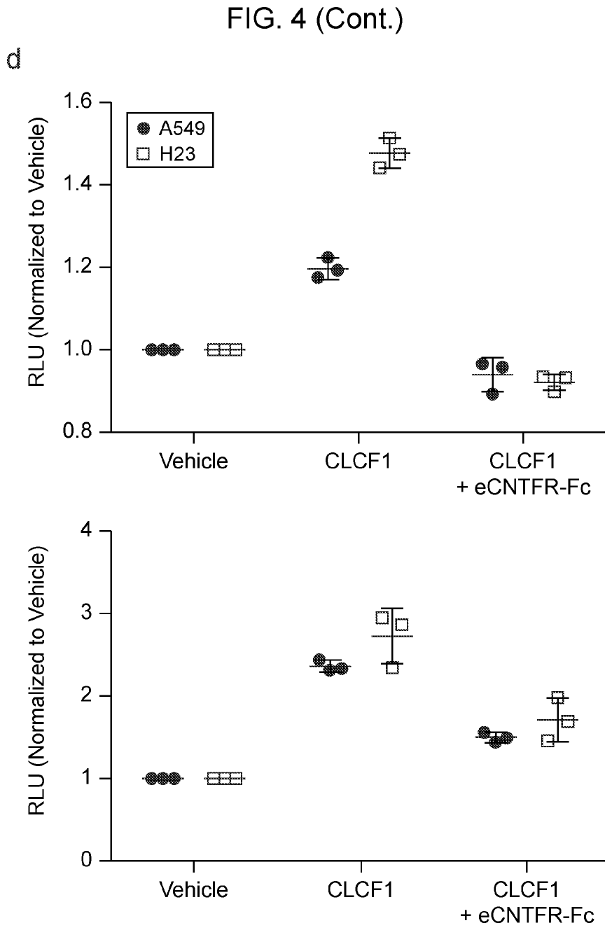

Demonstrated above is that CLCF1 expression is specifically prognostic of survival in patients with oncogenic KRAS-driven LUAD. As there are currently few therapeutic options for KRAS mutant tumors, developing new therapies for this subset is of particular clinical importance. To identify potential molecular determinants of response to eCNTFR-Fc, a panel of LUAD cell lines with a broad variety of genotypes was assembled and the effect of eCNTFR-Fc on cell viability was evaluated (FIG. 4, panel A). These cell lines exhibited a wide variety of sensitivities with the least sensitive (no effect) being normal lung cells (NL20) and the most sensitive being the LUAD cell line A549. The most sensitive cell lines were all KRAS mutant. Cell lines with wild-type KRAS or an EGFR mutation exhibited intermediate sensitivity. In contrast, H1755 and H1395 (both BRAF^{G469A}) cells were completely insensitive to CNTFR blockade. The BRAF^{G469A} mutation is a “Class 2” mutation that signals as constitutively active dimers and is expected to be independent of upstream KRAS signaling. Similarly, the two KRAS mutant cell lines carrying the Q61H mutation (FIG. 4, panel A) were completely insensitive to eCNTFR-Fc blockade. Q61H mutant KRAS lacks intrinsic GTPase activity and thus would also be expected to be insensitive to upstream signals that regulate GTPase activating proteins (GAPs). GAPs control the amount of GTP-bound KRAS in both KRAS mutants that retain GTPase hydrolysis and wild-type KRAS cells. Taken together, these results are consistent with a model in which CLCF1-CNTFR signals via gp130 to activate GAPs, which then regulate KRAS GTP binding and thus regulate downstream signals.

After engagement with the ligand, CNTFR activates gp130, which leads to activation of SHP2. In turn, SHP2 functions as a key upstream regulator of both oncogenic and wild-type KRAS through regulation of GTP loading. In both A549 and H23 LUAD cell lines, serum stimulation increased phosphorylation of P-SHP2, as well as P-STAT3 and P-ERK, as expected (FIG. 4, panels B and C). When cell lines were stimulated with recombinant CLCF1 in the absence of serum, phosphorylation levels of SHP2, STAT3, and ERK also increased, consistent with upstream signaling of CLCF1 serving to activate SHP2. Treatment with eCNTFR-Fc had a strong dampening of the effect of CLCF1 but was less effective in inhibiting the effect of full serum, which is to be expected since serum has other effects independent of the CLCF1-eCNTFR axis. To more directly establish the mechanistic link between the trimeric receptor complex and GTP loading of KRAS, directly measured were the levels of Ras-GTP in cells treated with recombinant CLCF1 and in the presence or absence of eCNTFR-Fc (FIG. 4, panel D). Levels of Ras-GTP increased after CLCF1 treatment and this effect was attenuated by eCNTFR-Fc. These results point to a link between CLCF1-CNTFR signaling and oncogenic KRAS and may explain why CLCF1 inhibition appears to be more effective in some KRAS genotypes but not others. Taken together these studies suggested that CLCF1 inhibition could be particularly effective in KRAS-mutant tumors as further explored below.

### Example 5—eCNTFR-Fc Sequesters CLCF1 and Inhibits In Vivo Tumor Growth

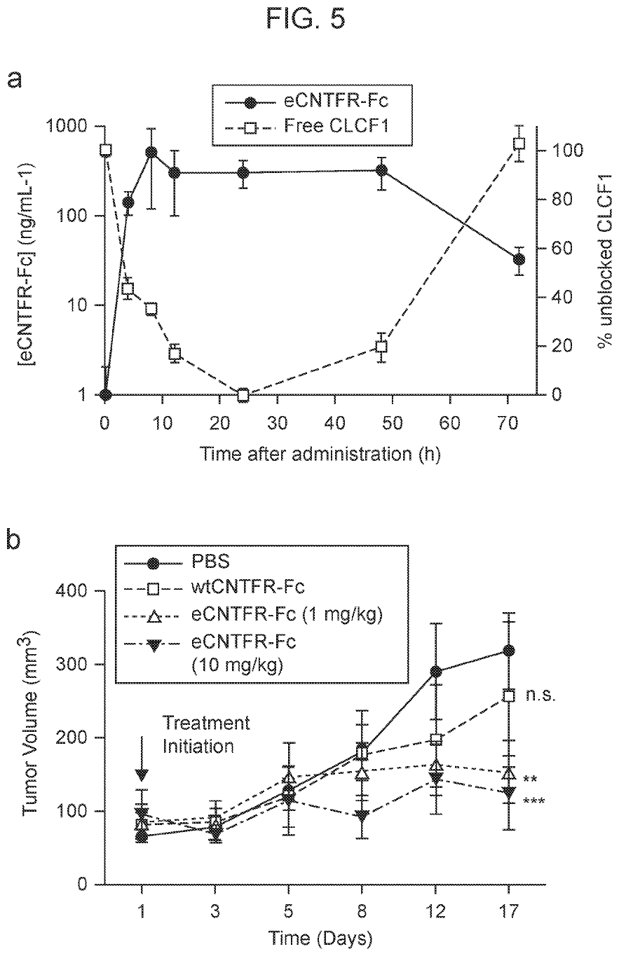

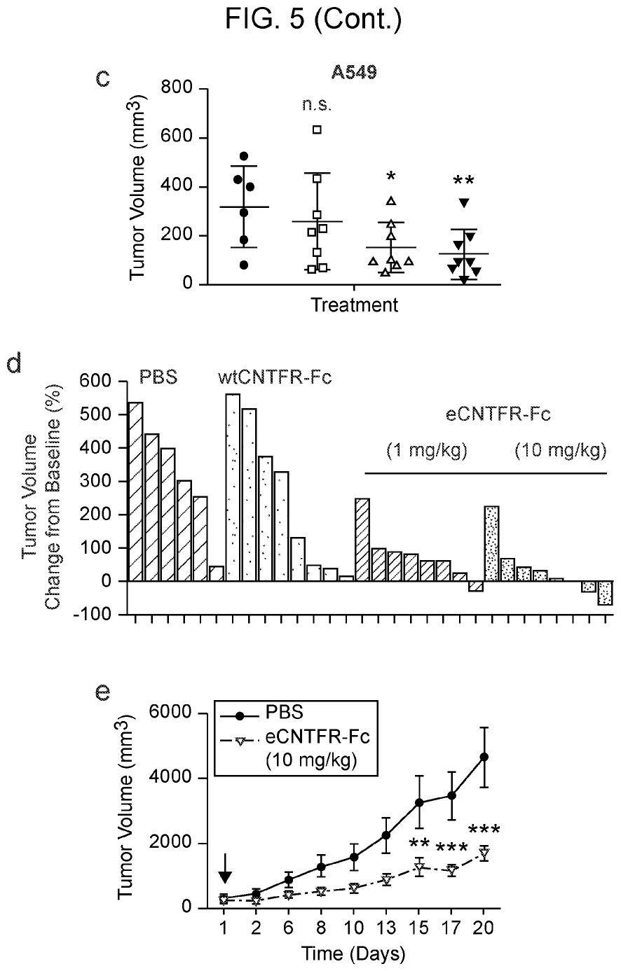

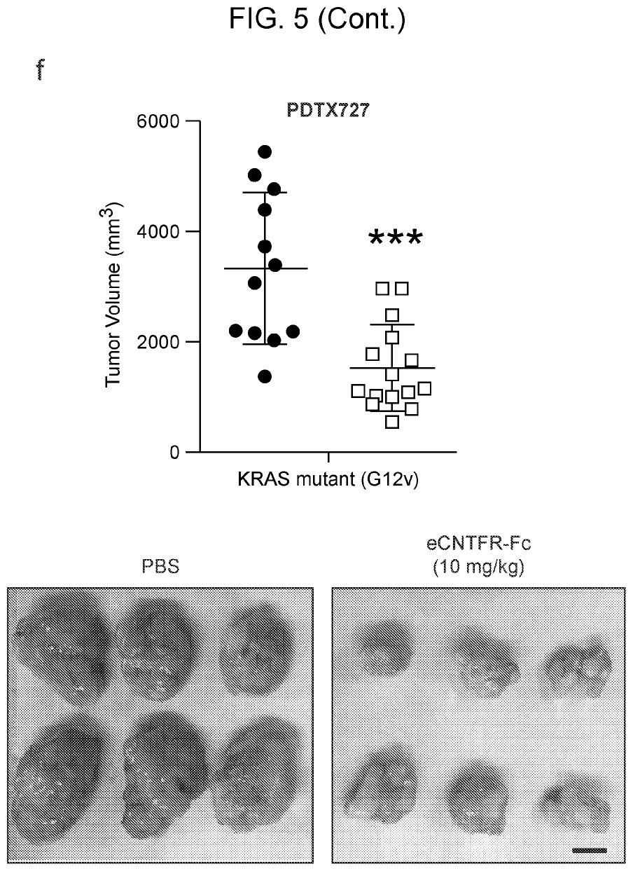

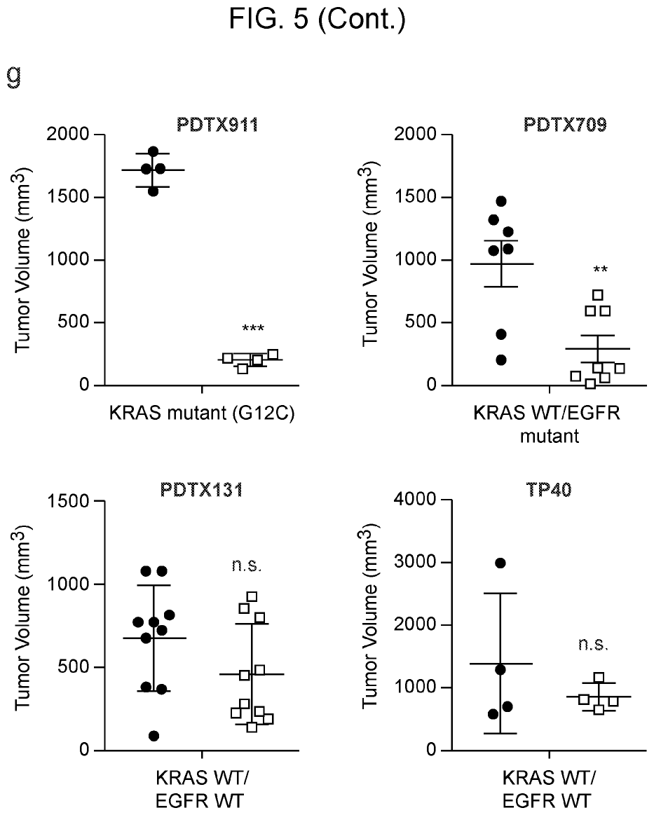

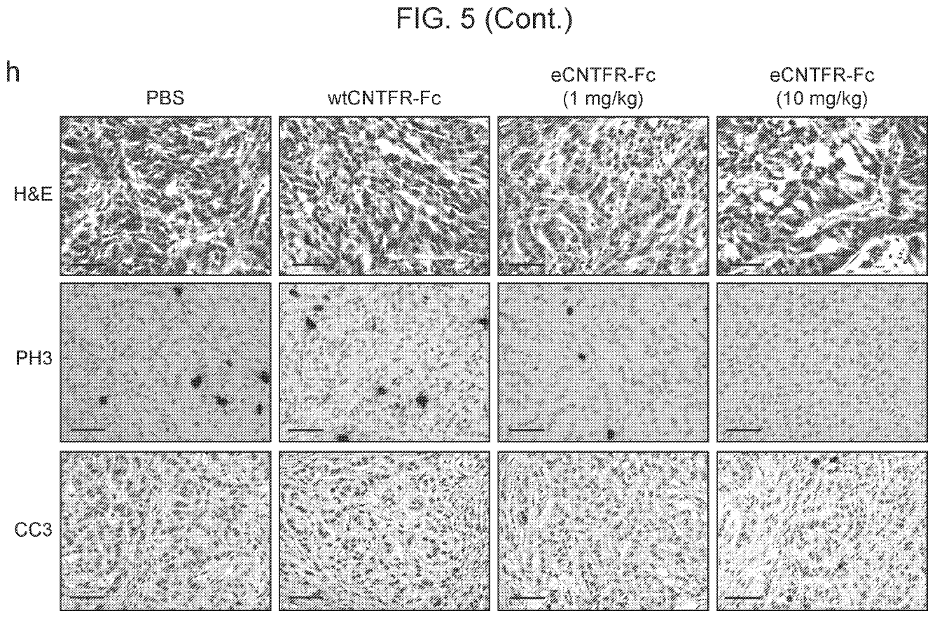

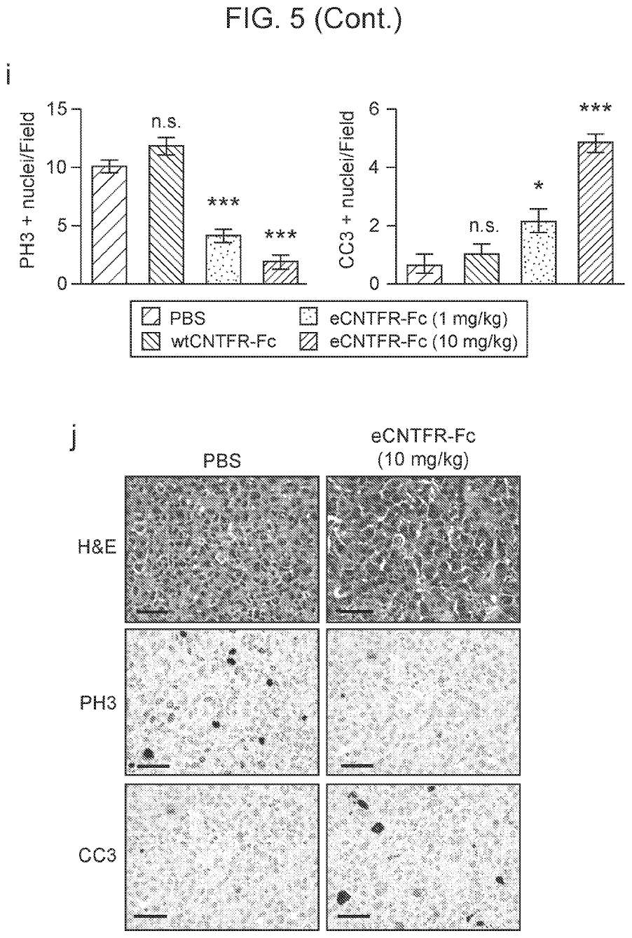

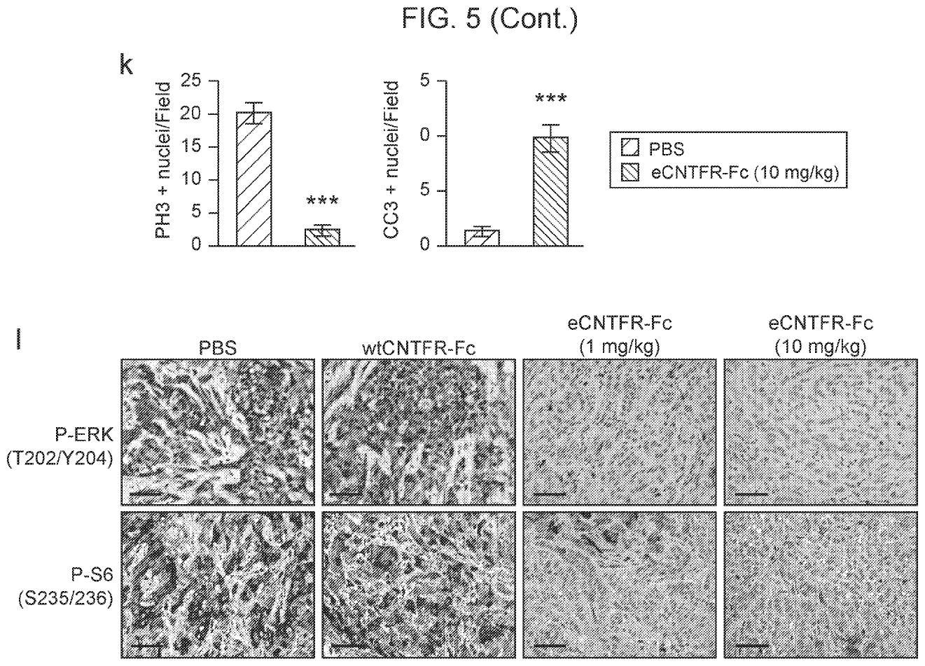

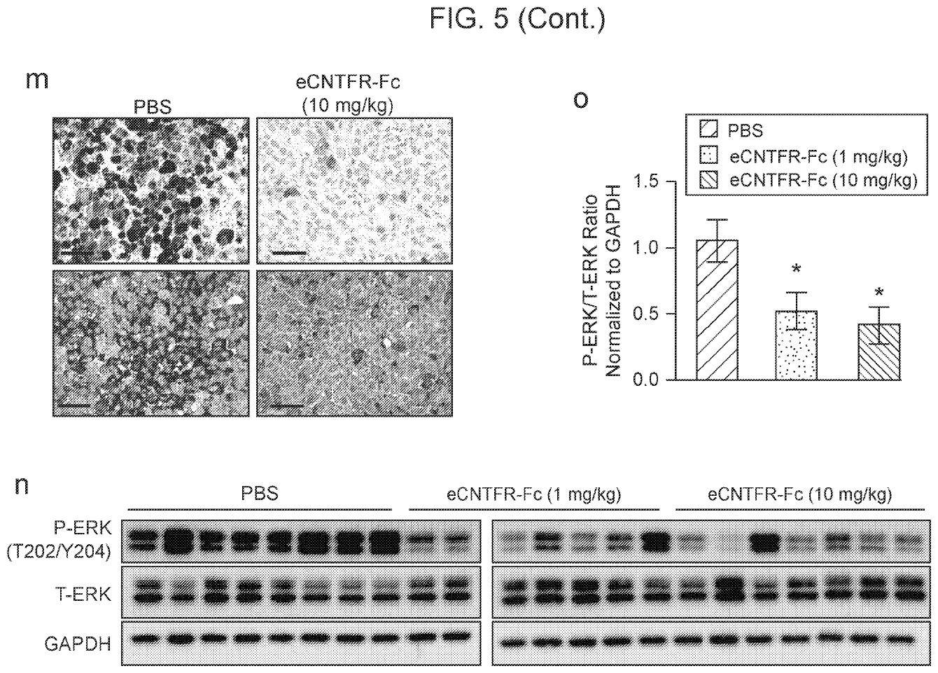

Next, the role of eCNTFR-Fc as an anti-tumor therapeutic in vivo was evaluated. To determine whether eCNTFR-Fc could effectively sequester mouse CLCF1, non-tumor bearing mice were treated with a single dose of eCNTFR-Fc. Serum levels of eCNTFR-Fc rapidly increased, along with a concomitant decrease in unbound CLCF1, which returned to baseline levels by 72 hours (FIG. 5, panel A). These results indicate that eCNTFR-Fc effectively binds to mouse CLCF1 and can reduce its availability in serum.

To test the therapeutic efficacy of eCNTFR-Fc, two LUAD cell lines were engrafted in immunodeficient mice and eCNTFR-Fc was dosed once tumors reached an average volume of 100 mm³. Treatment led to a dose-dependent tumor inhibition in both xenograft models (FIG. 5, panels B-D), whereas wtCNTFR-Fc had no effect. Evaluated next was the effect of eCNTFR-Fc on a panel of patient-derived xenograft tumors (PDTXs). Treatment with eCNTFR-Fc led to significant tumor growth inhibition in three of five LUAD PDTX models (FIG. 5, panels E-G). A significant decrease in proliferation markers and an increase in apoptosis were observed in both cell line xenografts and PDTX models (FIG. 5, panels H-K). The genotypes of the three PDTX models that responded to eCNTFR-Fc treatment were KRAS G12C, KRAS G12V, and EGFR mutant/KRAS wild-type (wt), while the non-responders were KRAS and EGFR wt. Also noted was that the CAFs with highest expression of CLCF1 were obtained from tumors with genotypes predicted to be most dependent on eCNTFR-Fc signaling.

As observed with CNTFR knock-down, treatment with eCNTFR-Fc also decreased activation of ERK (FIG. 5, panels L-O) and S6 Kinase (FIG. 5, panels L and M). To assess the time-dependent effect on signaling pathways, a short-term study was performed in which tumor-bearing mice were treated with eCNTFR-Fc and euthanized at different time points. These results suggest that eCNTFR-Fc first leads to inhibition of STAT3, which is then followed by delayed inhibition of ERK and S6 signaling.

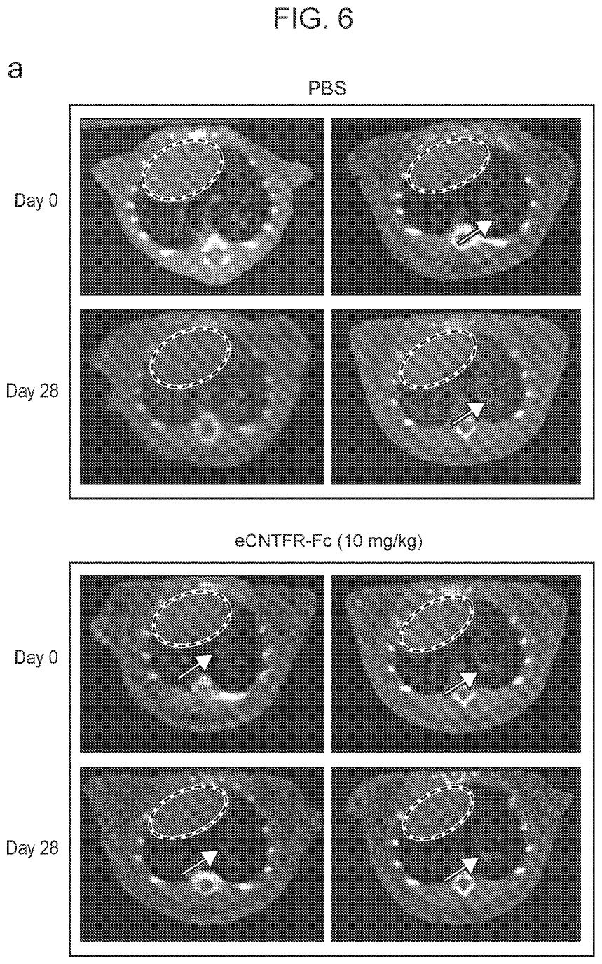

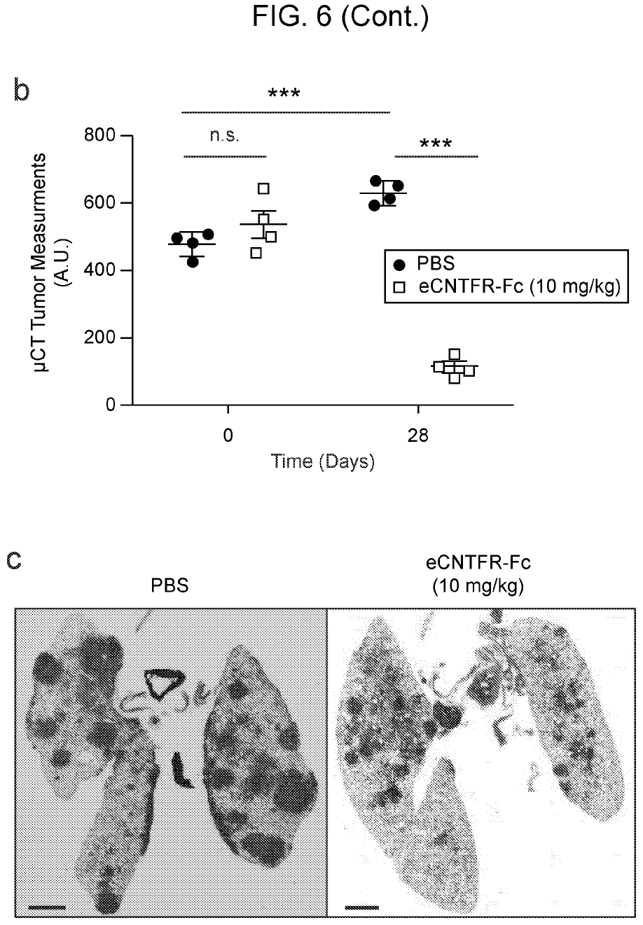

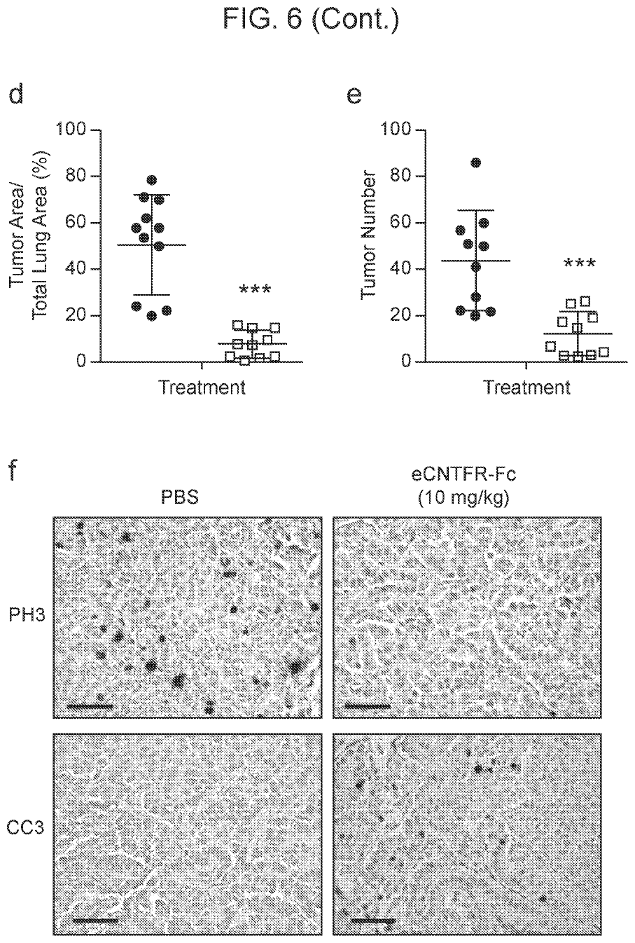

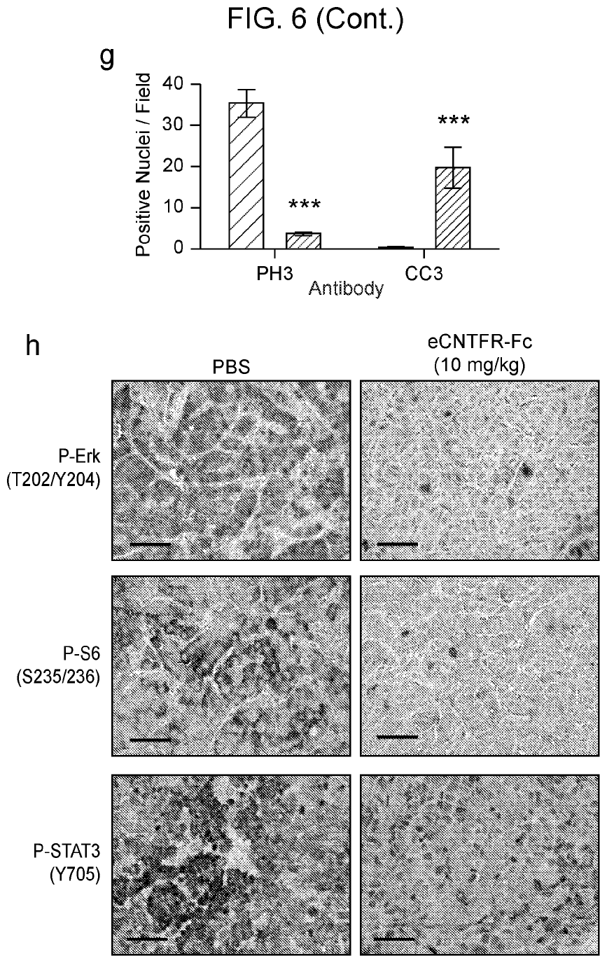

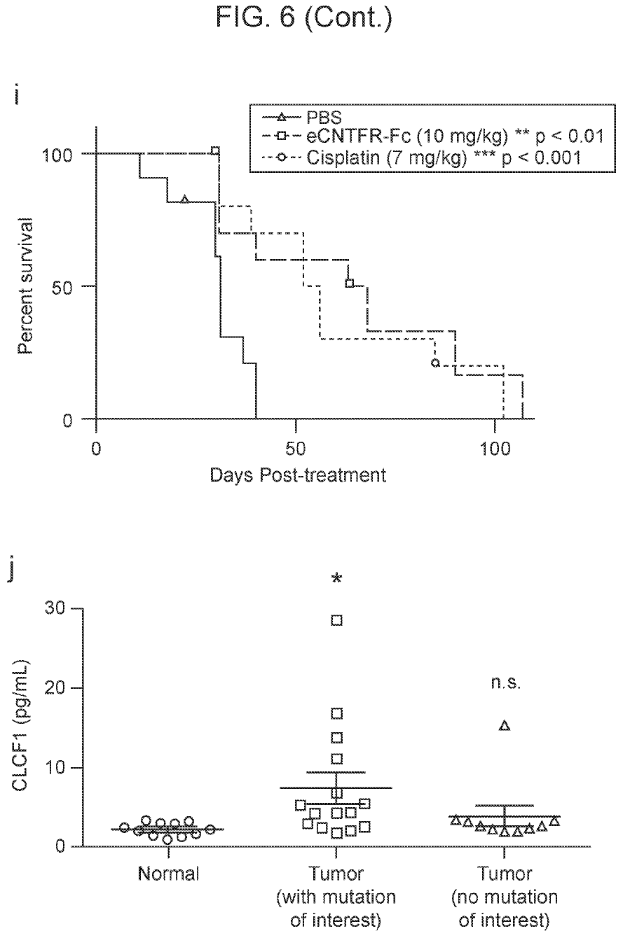

Next, these studies were extended to an autochthonous, highly aggressive genetically-engineered mouse (GEM) model of LUAD. Kras^{G12D}/Trp53^{f/f} mice treated with eCNTFR-Fc demonstrated decreased tumor burden compared to vehicle-treated controls (FIG. 6, panels A-E). Treatment with eCNTFR-Fc also led to decreased proliferation, increased apoptosis, and decreased activation of ERK, S6, and STAT3 signaling (FIG. 6, panels F-H). A survival assay comparing eCNTFR-Fc treatment with cisplatin was then performed. A platinum compound was chosen as a comparison as this is a commonly used standard chemotherapeutic human LUAD therapy (FIG. 6, panel I). Both cisplatin and eCNTFR-Fc treatment led to improved survival. However, cisplatin-treated mice had significant weight loss at the end of the study, whereas eCNTFR-Fc treated mice did not lose weight. Extensive evaluation of mouse tissues post-mortem in eCNTFR-Fc treated mice did not reveal any abnormalities, whereas platinum chemotherapy has been shown to induce significant adverse effects such as renal toxicity. These results strongly support the therapeutic efficacy of eCNTFR-Fc in LUAD.

Further development of eCNTFR-Fc as a bona fide therapeutic agent will be specifically enhanced by identification of an appropriate biomarker for activity of this pathway. A modest positive correlation between CLCF1 expression and decreased viability after treatment with eCNTFR-Fc was noted. While the data presented herein suggests that specific genotypes are more sensitive to eCNTFR-Fc, investigated next was whether CLCF1 levels in the plasma could also serve as an indicator of activity of this pathway in individual patients. A method to detect CLCF1 by ELISA was developed, with eCNTFR-Fc acting as a capture agent, and used to measure the levels of CLCF1 in the plasma of cancer patients. A trend towards higher levels of CLCF1 in LUAD patients relative to healthy controls was observed. Moreover, patients with genotypes sensitive to eCNTFR-Fc (with ‘mutation of interest’) had significantly higher levels of CLCF1 than those without the mutation of interest (FIG. 6, panel J). The data was analyzed further using logistic regression (logit) to demonstrate whether CLCF1 in the blood can predict if a tumor has a particular mutation of interest (KRAS G12C, KRAS G12V, or KRAS wt/EGFR mutant) [Odds ratio: 8.35 (CI 95% 6.36-10.33); p-value: 0.04]. Taken together, these results indicate that CLCF1 plasma concentration combined with genotypic analysis of the tumor serve as useful biomarkers for selection of patients most likely to have therapeutic benefit from eCNTFR-Fc.

### Methods

Lung Adenocarcinoma Mouse Model

Lox-stop-Lox-Kras^{G12D} (129 Sv/Jae), Trp53^{fl/fl} (FVB), and Rosa26-LSL-tdRFP (C57BL/6J) mice were maintained in a virus-free environment. Mice were intra-nasally infected with 5×10⁶ pfu of adenovirus expressing Cre (University of Iowa) at eight- to ten-weeks of age. Mice were dosed with eCNTFR-Fc (10 mg/kg) or PBS (vehicle) by intraperitoneal injection for four weeks three times per week beginning eight-weeks post-infection. Mice were weighed at the beginning of study and periodically throughout drug treatment.

Human LUAD Survival and Gene Expression Analysis

CLCF1 TPM log₂ expression for the cohorts (LUAD; LUSC) were downloaded directly from the Broad Institute with R programming language using the package FirebrowseR (1.1.35). We used only expression data categorized as either TP (Primary Tumor) or NT (Normal). The full LUAD expected counts (RSEM level 3) was downloaded directly from the FIREHOSE Broad GDAC website. Somatic mutation for the LUAD data set was acquired from the UCSC Xena public repository. Only samples with a non-silent KRAS mutation(s) were associated with the KRAS mutation group; samples with KRAS silent mutations were not included as the KRAS wild-type group and were excluded from the analysis. Clinical data for LUAD survival analysis including censored data such as overall survival was acquired from published clinical aggregation of the TCGA dataset. Survival analysis curves and multivariate cox hazard regression was completed in R using the survminer (0.4.3.999) and survival package (2.44-1.1), respectively. For Cox regression analysis we adjusted for age of diagnosis, gender, and cancer stage. We grouped samples (Normal vs High) based on the quantile of the respected gene expression: normal is <75th percentile and high is >75^{th} percentile.

Quantitative Reverse Transcriptase—PCR

RNA was isolated using TRIzol reagent (Invitrogen) and further purified using Qiagen miniRNA columns (Qiagen). cDNA was generated with a DyNAmo cDNA Synthesis Kit (New England Biolabs) and quantitative reverse transcriptase-PCR (qRT-PCR) was performed using SYBRGreen (Applied Biosystems; see Supplementary Table 5 for primer sequences). qRT-PCR was performed as follows: 95° C. for 10 min, 35 cycles of 95° C. for 15 s and 60° C. for 1 min.

Generation of Patient-Derived Tumor Xenografts (PDTXs)

Fresh patient samples were cut into 1×1 mm fragments and either implanted fresh or frozen in 90% FBS, 10% DMSO for later use. Tumor fragments were dipped in Matrigel (Corning Matrigel #356234) and implanted in the subrenal capsule of NOD scid gamma (NSG) mice. Successfully implanted tumors were harvested at ˜1-2 cm. A fragment was kept for histology and the remainder was digested with collagenase for 45 min at 37° C. and filtered through 70 μm filter. For RNA/DNA isolation, cells were depleted of mouse stroma (using antibodies against Ter119, CD45, CD31, and mouse MHC class I) on a MACS column (Miltenyi Biotech). For subsequent passages and drug studies, cells were implanted subcutaneously in flanks of NSG mice (5×10⁵ cells per flank) in 100 μL α-MEM and 20 μL Matrigel (Corning). Xenograft tumor fragments were stored at −80° C. until use.

Cells were passed through 100 μm and 40 μm cell strainers and centrifuged for 1,200 rpm for 8 min. Cells were incubated in RBC lysis buffer and resuspended in 6 ml of media and spun through 0.5 ml of serum layered on the bottom of the tube to remove cellular debris. Cells were depleted of lineage-positive cells using biotin conjugated anti-mouse CD45, CD31 and Ter119 (eBiosciences) and depleted on a MACS LS column (Miltenyi Biotec). 5×10⁵ single cells were mixed with Matrigel (BD Biosciences) and injected into the flanks of 6- to 8-week-old female NSG mice. Tumor volume was measured at the times indicated and calculated using the ellipsoid formula [0.5(length×width²)].

Serum Analysis and Toxicity Studies

Blood samples from individual mice were collected at the end of the experiment under terminal anesthesia using cardiac puncture. Serum was separated from blood within 1 hr by centrifugation at 500 g for 10 min. Samples were aliquoted and stored at −80° C. for subsequent testing. Comprehensive Metabolic Panel (CMP) and Complete Blood Count (CBC) were done by the Animal Diagnostic Laboratory at Stanford Veterinary Service Center. Toxicity studies including necropsy and comprehensive histopathological analysis of each organ were performed by a veterinary pathologist.

Treatment of Mice with eCNTFR-Fc

When tumors reached an average size of 100 mm³ per tumor, mice were stratified into treatment arms based on average tumor size per group. Mice were then dosed with eCNTFR-Fc (10 mg/kg) or PBS (vehicle) by intraperitoneal injection for two to four weeks three times per week. Mice were weighed at the beginning of study and periodically throughout drug treatment. Tumor volume was measured with digital calipers three to four times per week.

Knockdown Studies in Xenografts

pLKO shRNA constructs were purchased from Thermo Fisher Scientific. Lentivirus for each construct was generated by transfecting 293 cells with polyethylenimine (PEI), viral supernatants were collected on days 1 and 2 after transfection and pooled on day 2. Viral supernatants were then filtered through 0.45 μM PES filters. Viral pellets were re-suspended on a platform rocker for 2 h with ˜500 uL fresh media. Cells were dissociated into a single cell suspension using Collagenase (Sigma) digestion buffer and filtered through a 70 μM filter and depleted for lineage (as above) on a MACS column. The resulting cell suspension was then plated at approximately 5×10⁶ cells per well of a 6-well plate and spin infected with polybrene (Sigma) and virus in media at 1500 rpm at room temperature for 30 min (Sorvall XRT centrifuge) followed by incubation at 37° C. After selection with puromycin (2 μg/mL), cells were trypsinized, filtered and counted for viable cells. Cells were then implanted (as above) keeping the viable cell count consistent between study groups. Remaining cells were kept for confirmation of gene knockdown.

Cell Extracts and Western Blot Analysis

For Total Cell Extracts, Cells were Lysed Using NP-40 Lysis Buffer (20 mM Tris-HCl, pH 8.0, 137 mM NaCl, 10% Glycerol, 1% NP-40, dH₂0, 1× protease inhibitors (Sigma P8349-1ML) and 1× phosphatase inhibitor cocktail (Sigma P5726-1ML) for 15 minutes, sonicated and lysed for 30 minutes. Tumors were thawed and mechanically disrupted using the Bio-Gen PRO200 Homogenizer (PRO Scientific) on ice prior to lysis. Protein concentration was determined by BCA assay (Thermo Fisher). Proteins were resolved by SDS-PAGE, transferred to a PVDF membrane and analyzed by Biorad Chemi Doc apparatus. Antibodies used were as follows: P-AKT (#4060, Cell Signaling, 1:1000), T-AKT (#75692, Cell Signaling, 1:1000), P-ERK1/2 (#4370, Cell Signaling, 1:1000), T-ERK1/2 (#4695, Cell Signaling, 1:1000), P-STAT3 (#9145, Cell Signaling, 1:1000), T-STAT3 (#9139, Cell Signaling, 1:1000), GAPDH (#9485, Abcam, 1:1000).

Histology and Immunohistochemistry

Tissue specimens were fixed in 10% buffered formalin for 24 h and stored in 70% ethanol until paraffin embedding. 5 μm sections were stained with hematoxylin and eosin (HE) or used for immunohistochemical studies. Immunohistochemistry was performed on formalin-fixed, paraffin embedded mouse and human tissue sections using a biotin-avidin method. The following antibodies were used (at indicated dilutions): P-Akt (#4060, Cell Signaling, 1:100), P-ERK1/2 (#4370, Cell Signaling, 1:400), P-Histone H3 (#9701, Cell Signaling, 1:200), Cleaved Caspase 3 (#9661, Cell Signaling, 1:200), CNTFR (#175387, Abcam, 1:50). Sections were developed with DAB and counterstained with hematoxylin. Analysis of the tumor area and IHC analysis were done using ImageJ software by measuring pixel units.

Cell Assays

Cell Viability: Cells were seeded in 96-well plates at 2,000 cells per well (optimal density for growth) in a total volume of 100 μL media containing 10% Bovine Growth Serum (BGS). After 24 h incubation, cell viability was assessed by AlamarBlue® assay (Thermo Fisher) for 7 days according to the manufacturer's instructions.

Colony-formation assay: For long-term colony-formation assay, 10,000-50,000 cells per well were seeded in 6-well plates. After 12 days, cells were fixed with methanol, stained with crystal violet, photographed, and quantified.

3D Spheroid methylcellulose assay: For anchorage-independent sphere growth the cells were seeded into 24-well ultra-low attachment plates (20,000 viable cells per well) in 2 mL of complete medium supplemented with 0.5% methylcellulose. The spheres were allowed to form for 9-20 days (depending on the cell line). Spheres were imaged with Leica DMi8 microscope (brightfield). Sphere size and number were quantified using ImageJ.

Analysis of Ras-GTP Levels

Levels of activated Ras-GTPase were determined using the Ras GTPase ELISA Kit (Abcam 134640) per the manufacturer's instructions, similar to a previously published method. Briefly, 1×10⁶ cells were seeded in RPMI media supplemented with 10% bovine growth serum and 1% penicillin/streptomycin in 10-cm tissue culture dishes and incubated at 37° C. in 5% CO₂ until cells reached 60% confluence. Cells were then serum starved with RPMI and 1% penicillin/streptomycin for 24 h. Cells were subsequently incubated in CLCF1 (10 nM) and eCNTFR-Fc (2.5 μM) for 20 min at 37° C. in 5% CO₂. Media was then removed and cells were washed once in ice-cold PBS and processed following the manufacturer's protocol.

Statistics

Kaplan-Meier survival curves were calculated using the survival time for each mouse from all littermate groups. The log-rank test was used to test for significant differences between the groups. For image quantification and gene expression analysis, statistical significance was assayed by Student's t-test with Prism GraphPad software (two-tailed unpaired or paired t-test depending on the experiment—variance was first systematically examined using an F-test for both One-way combined with Dunnett's multiple correction test and Two-way ANOVA depending on the experiment). * P<0.05; ** P<0.01; *** P<0.001. Data are represented as mean±S.D. for in vitro experiments and mean±S.E.M. for in vivo experiments. In boxplots, box represents 25^{th} and 75^{th} percentiles with midline indicating the median; whiskers extend to the lowest/highest value within 1.5 times the interquartile range.

Logistic Regression Model

Table created using Stargazer v.5.2.2 by Marek Hlavac, Harvard University. Model contains only blood CLCF1 levels (pg/mL) and no other covariates were used.

Recombinant CLCF1 Production

cDNA encoding for CLCF1 without the signal peptide sequence (28-225) was cloned into pET28b plasmid with inducible lac promoter using BsaI and XhoI restriction sites and amplified in DH10B cells. For expression, purified plasmids were transformed into Rosetta gami cells. Inclusion bodies were solubilized in 60% ddH₂O, 40% acetonitrile, 0.1% TFA containing 5 mM DTT. Reversed-phase high-performance liquid chromatography (RP-HPLC) was used to purify CLCF1. Protein purity was further analyzed using SDS-PAGE and quantified using a Nanodrop 2000 (Thermo Scientific). A value of 39,549 M⁻¹cm⁻¹ was used as the extinction coefficient to quantify protein concentration.

Soluble CNTFR, LIFR, and Gp130 Production

cDNA corresponding to the extracellular domains of CNTFR (1-342), LIFR (1-534), and gp130 (1-619) was cloned into the pAdd2 plasmid and amplified in DH10B cells. For expression, purified plasmids were transfected into human HEK 293 cells using PEI (#23966-2, Polysciences). Briefly, PEI was dissolved in dH₂O to 1 g/L. For 500 mL transfection volume, 0.5 mg of purified DNA and 1 mL of PEI was dissolved in 10 mL of OptiPro Serum Free Media (#12309-019, Thermo Fisher Scientific) each, then mixed immediately. After 15 min the solution was added dropwise to 500 mL of cells. The cells were incubated on a rotary shaker at 120 RPM in a humidified incubator at 37° C. and 5% CO₂. Fc fusion proteins were purified using a protein A (#101142, Fisher Scientific) affinity column; proteins containing a hexahistidine tag were purified using a nickel-NTA (#30210, Qiagen) affinity column. Proteins were then further purified using size exclusion chromatography. The following extinction coefficients were used for protein quantification: CNTFR variants: 70,275 M⁻¹cm⁻¹; CNTFR-Fc variants: 206,410 M⁻¹cm⁻¹; gp130: 130,470 M⁻¹cm⁻¹; gp130-Fc: 326,800 M⁻¹cm⁻¹; LIFR: 98,610 M⁻¹cm⁻¹; and LIFR-Fc: 263,080 M⁻¹cm⁻¹.

Generation and Screening of a CNTFR Library Created Via Error-Prone PCR

CNTFR was expressed in yeast as a genetic fusion to the agglutinin mating protein Aga2p. cDNA encoding the human CNTFR extracellular domain (residues 18-342) was cloned into the pCTCON2 yeast display plasmid using NheI and BamHI restriction sites. An error-prone library was created using the CNTFR extracellular domain as a template, and mutations were introduced by using Taq polymerase (#50-811-694, Fisher Scientific) and 55 mM MgCl₂. Separate PCR reactions were performed using different concentrations of MnCl₂ (0, 0.01, 0.05, 0.1, and 015 mM). Products from these reactions were purified using gel electrophoresis. Purified mutant cDNA and linearized plasmid were electroporated into EBY100 yeast, where they were assembled in vivo through homologous recombination. Library size was estimated to 8.1×10⁷ by dilution plating and colony counting.

Yeast displaying high-affinity CNTFR variants were isolated using fluorescence-activated cell sorting (FACS) using a BD Aria II flow cytometer (Stanford FACS Core Facility) and analyzed using a BD FACSCalibur. Screens were carried out using equilibrium binding conditions where yeast were incubated at room temperature in phosphate-buffered saline containing 1 mg/mL BSA (PBSA) with the following CLCF1 concentrations: for sort 1, 20 nM CLCF1 for 3 h; for sort 2, 2 nM CLCF1 for 6 h; for sort 3, 0.5 nM CLCF1 for 12 h. After incubation with CLCF1, yeast were pelleted, washed and resuspended in PBSA with 1:500 ratio of chicken anti-c-Myc (#A21281, Invitrogen) for 30 min at 4° C. Yeast were then washed and pelleted, and secondary labeling was performed on ice for 30 min using PBSA with 1:100 dilution of goat anti-chicken PE (#sc-3730, Santa Cruz Biotech) and mouse anti-HIS Hilyte Fluor 488 (#61250-H488, Anaspec).

Sorted clones were propagated and subjected to further rounds of FACS. After the last round of screening plasmid DNA was recovered using a Zymoprep kit (#50-444-107, Zymo Research Corp), transformed into DH10B electrocompetent cells, and isolated using plasmid miniprep kit. Sequencing was performed by Molecular Cloning Laboratories. Samples were analyzed on a FACSCalibur (BD Biosciences), and data were analyzed using FlowJo software (Treestar Inc).

Generation and Screening of a CNTFR Library Created Via Staggered Extension Process (StEP)

The StEP method was performed as described previously and the resulting library was displayed on yeast. Briefly, 20 unique sequences were selected randomly from the yeast population isolated from the final sort round of the error-prone PCR library. 1 ng of each of the templates was combined and 20 ng total template was mixed with the final concentrations of 0.15 μM each primer, 1×PCR buffer, 200 μM dNTP mix, 1.5 mM MgCl₂, and 2.5 U Taq polymerase in sterile dH₂O to 50 μL. The extension protocol was run for 100 cycles using the following parameters: 94° C. for 30 s (denaturation) and 55° C. for 10 s. Products from these reactions were purified using gel electrophoresis. Purified mutant cDNA and linearized plasmid were electroporated in EBY100 yeast, where they were assembled in vivo through homologous recombination. Library size was estimated to 7.9×10⁷ by dilution plating.

Screens were performed using a single round of equilibrium binding sorting followed by two rounds of kinetic off-rate sorts. For kinetic off-rate sorts, yeast were incubated with 2 nM CLCF1 for 2 h at room temperature, after which cells were washed twice to remove excess unbound CLCF1 and resuspended in PBSA containing 20 nM wtCNTFR-Fc to prevent rebinding of dissociated CLCF1. For the length of the unbinding steps, 10 h was used for sort 2, and 24 h was used for sort 3. Libraries were stained to detect CLCF1 binding and c-myc expression as described above and sorts were conducted such that the 0.5-1% of clones with the highest CLCF1 binding/c-Myc expression ratio were collected by FACS, enriching the library for clones with the highest binding affinity to CLCF1. Plasmid DNA was isolated and sequenced as described above.

Library Generation and Screening for CNTFR Variants that do not Bind LIFR

To engineer CNTFR variants with decreased binding for LIFR, error-prone PCR was used to introduce random mutations into CNTFR variant 4, creating a library with an estimated diversity of about 1×10⁸ transformants. The resulting library was displayed as fusion proteins on the yeast cell surface and screened to isolate the population with decreased binding signal for LIFR-Fc in the presence of CLCF1. To retain the binding affinity for CLCF1, screening was performed by alternating between positive selection for 0.5 nM CLCF1 and negative selection for increasing concentrations of LIFR-Fc. After six rounds of sorting, two consensus mutations emerged (Y177H and K178N). These mutations additively contributed to decreased LIFR binding.

Yeast-Displayed CNTFR Binding Assays

Yeast displaying the CNTFR constructs were incubated with varying concentrations of CLCF1 for 12 h at room temperature to reach equilibrium binding. This was followed by washing with PBSA and resuspension in PBSA with 1:500 ratio of chicken anti-c-Myc antibody for 30 min at 4° C. Yeast were then washed and pelleted, and secondary labeling was performed on ice for 30 min using PBSA with 1:100 dilution of goat anti-chicken PE antibody and mouse anti-HIS Hilyte Fluor 488 antibody. Then samples were washed and analyzed by flow cytometry using BD Accuri flow cytometer. Samples were analyzed on BD Bioscience software, and data were analyzed using FlowJo software (Treestar Inc).

For assays to detect binding with the β receptors, varying concentrations of LIFR constructs and/or gp130 constructs with 10 nM CLCF1 were added to yeast-displayed CNTFR. For His-tagged constructs, mouse anti-HIS Hilyte Fluor 488 antibody was used to detect binding. For detecting Fc-fusion constructs, anti-mouse-Fc Alexa 488 antibody (#A11029, Thermo Fisher) was used.

Cell-Free Binding Assays

96-well plates were coated with 10 μg/mL of anti-HIS antibody or anti-mouse-Fc antibody overnight and blocked with 5% milk for 1 h. The plates were then washed twice with PBSA. Varying concentrations of soluble CNTFR-HIS or CNTFR-Fc fusion constructs were incubated with 2 nM CLCF1 in PBSA for 12 h at room temperature. The mixture was then added to 96-well plates coated with anti-HIS antibody or anti-mouse-Fc antibody respectively for 1 h followed by washing twice with BPBS. Subsequently, the wells were incubated with 1:1000 diluted anti-CLCF1 rabbit antibody (#ab26125, Abcam) for 2 h at room temperature then washed four times with PBS. The wells were incubated with 1:1000 diluted HRP conjugated anti-rabbit antibodies (#111-035-144, Jackson ImmunoResearch) for 2 h at room temperature, washed four times with PBS. 1-Step Ultra TMB ELISA (#34029, Thermo Fisher Scientific) was used for the readout.

Phosphorylation Assays

A549 or H23 cells were grown until 50% confluence in 6-well plates. The cells were incubated in CLCF1 (10 nM) and CNTFR constructs (10 nM) for 20 min at 37° C. in 5% CO₂, then lysed with NP-40 buffer containing protease inhibitor (#P8340, Sigma Aldrich) and phosphatase inhibitor (#P5726, Sigma Aldrich). Equal amounts of lysate were loaded on Bis-Tris gels and transferred onto nitrocellulose membrane. Western Blot analysis was performed with the reagents above. Chemiluminescence was detected using the ChemiDoc XRS System (Bio-Rad). NP-40 buffer was composed of 20 mM Tris pH 8.0, 137 mM NaCl, 10% glycerol, and 1% IGEPAL/NP40.

CLCF1 Cell Proliferation Assay

5×10³ A549 and H23 cells were seeded and grown for 24 h, and then serum starved by incubating for 24 h in DMEM with 0.1% BSA. CLCF1 and CNTFR constructs were then added and incubated for 72 h at 37° C./5% CO₂. Next, AlamarBlue reagent (#DAL1025, Fisher Scientific) was added to each well and incubated for 1 h at 37° C./5% CO₂. The cell metabolic activity was detected by measuring fluorescence using 560EX nm/590EM nm. Error bars represent the standard deviation of triplicate wells. Data was measured against negative control with only media.

Analysis of In Vivo CLCF1 Sequestration of eCNTFR-Fc

Non-tumor bearing NSG mice were administered a single dose of eCNTFR-Fc at 10 mg/kg body weight via intraperitoneal injection. The doses were formulated in 200 μL volume. Two mice were analyzed per condition, and untreated mice were used to determine baseline CLCF1 levels. Terminal blood collection was done at euthanasia by cardiac puncture at 6 h, 12 h, 24 h, 36 h, 48 h, and 72 h after injection, and serum was isolated for analysis. CLCF1 levels were measured using a sandwich ELISA. In this assay, eCNTFR-Fc was used as a capturing agent to ensure the detection of free, unbound CLCF1. 96-well plates were coated with 10 μg/mL of eCNTFR-Fc overnight at room temperature and blocked with 5% milk. After the coated plates were washed twice with PBSA, the plates were incubated with the collected serum at room temperature for 2 hours. After the plates were washed with BPBS twice, detection of CLCF1 was carried out using polyclonal anti-CLCF1 antibody and anti-rabbit HRP. After washing the plates 4 times with BPBS, ELISAs were developed using the 1-Step Ultra TMB ELISA.

Kim et al. (2019) Nature Medicine 25:1783-1795, including but not limited to any of the methods, data, agents (e.g., engineered CNTFR ligands), reagents, etc. disclosed therein constitutes a part of the present disclosure and is incorporated herein in its entirety for all purposes.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of treating a KRAS mutant cancer in an individual, comprising:
administering to an individual identified as having a KRAS mutant cancer a therapeutically effective amount of an agent that inhibits cardiotrophin-like cytokine factor 1 (CLCF1)-ciliary neurotrophic factor receptor (CNTFR) signaling.

2. The method according to claim 1, wherein the KRAS mutant cancer is a KRAS mutant lung cancer.

3. The method according to claim 2, wherein the KRAS mutant lung cancer is a KRAS mutant non-small cell lung cancer (NSCLC).

4. The method according to claim 3, wherein the KRAS mutant NSCLC is a KRAS mutant lung adenocarcinoma (LUAD).

5. The method according to claim 1, wherein the KRAS mutant cancer is a KRAS mutant pancreatic cancer.

6. The method according to claim 5, wherein the KRAS mutant pancreatic cancer is a KRAS mutant pancreatic ductal adenocarcinoma (PDAC).

7. The method according to any one of claims 1 to 6, wherein the agent is administered to an individual identified as having a KRAS mutant cancer comprising an amino acid substitution at position 12 of human KRAS, and wherein numbering is as in SEQ ID NO:1.

8. The method according to claim 7, wherein the agent is administered to an individual identified as having a KRAS mutant cancer comprising an amino acid substitution selected from the group consisting of: G12A, G12C, G12D, G12S , and G12V.

9. The method according to claim 8, wherein the agent is only administered to an individual identified as having a KRAS mutant cancer comprising an amino acid substitution selected from the group consisting of: G12A, G12C, G12D, G12S , and G12V.

10. The method according to any one of claims 1 to 9, further comprising, prior to administering the agent, identifying the individual as having the KRAS mutant cancer.

11. The method according to any one of claims 1 to 9, further comprising, prior to administering the agent, determining that the individual has a KRAS mutant cancer.

12. The method according to claim 11, wherein determining that the individual has a KRAS mutant cancer comprises genotyping cancer cells obtained from the individual, wherein the genotyping indicates that the cancer cells are of a KRAS mutant cancer.

13. The method according to claim 12, wherein the genotyping comprises sequencing at least a portion of a gene or transcript encoding KRAS.

14. The method according to claim 12, wherein the genotyping is by polymerase chain reaction (PCR).

15. The method according to any one of claims 1 to 14, further comprising, prior to administering the agent, determining the plasma concentration of CLCF1 in the individual.

16. The method according to any one of claims 1 to 15, wherein the agent specifically binds CNTFR and inhibits signaling through CNTFR.

17. The method according to any one of claims 1 to 16, wherein the agent specifically binds CNTFR and inhibits interaction between CNTFR and CLCF1.

18. The method according to any one of claims 1 to 16, wherein the agent specifically binds CNTFR or a ligand-CNTFR complex subunit and inhibits interaction between CNTFR and the ligand-CNTFR complex subunit.

19. The method according to claim 18, wherein the ligand-CNTFR complex subunit is glycoprotein 130 (gp130) or leukemia inhibitory factor receptor (LIFR).

20. The method according to claim 16, wherein the agent is an engineered CNTFR ligand selected from the group consisting of:
an engineered CNTFR ligand that exhibits increased binding affinity for CNTFR relative to the corresponding wild-type CNTFR ligand,
an engineered CNTFR ligand that results in reduced binding affinity of gp130, LIFR, or both, for a complex comprising the engineered CNTFR ligand and CNTFR, relative to the binding affinity for a complex comprising the corresponding wild-type CNTFR ligand and CNTFR, and
an engineered CNTFR ligand that exhibits increased binding affinity for CNTFR relative to the corresponding wild-type CNTFR ligand and results in reduced binding affinity of gp130, LIFR, or both, for a complex comprising the engineered CNTFR ligand and CNTFR, relative to the binding affinity for a complex comprising the corresponding wild-type CNTFR ligand and CNTFR.

21. The method according to claim 20, wherein the engineered CNTFR ligand is an engineered CLCF1 that binds to CNTFR and includes an amino acid substitution selected from L86F, Q96R, H148R, and any combination thereof, wherein numbering is as in SEQ ID NO:6, and wherein the engineered CLCF1 comprises 70% or greater, 75% or greater, 80% or greater, 85% or greater, 90% or greater, 95% or greater, 99% or greater, or 100% amino acid sequence identity to the amino acid sequence set forth in SEQ ID NO:6.

22. The method according to claim 20, wherein the engineered CNTFR ligand is an engineered CLCF1 that binds to CNTFR and includes an amino acid substitution selected from Y22C, L86F, Q96R, H148R, F151A, K154A, W169L, K180R, and any combination thereof, wherein numbering is as in SEQ ID NO:7, and wherein the engineered CLCF1 comprises 70% or greater, 75% or greater, 80% or greater, 85% or greater, 90% or greater, 95% or greater, 99% or greater, or 100% amino acid sequence identity to the amino acid sequence set forth in SEQ ID NO:7.

23. The method according to any one of claims 1 to 15, wherein the agent specifically binds CLCF1 and inhibits signaling through CNTFR.

24. The method according to claim 23, wherein the agent specifically binds CLCF1 and inhibits interaction between CLCF1 and CNTFR.

25. The method according to claim 23 or claim 24, wherein the agent is a soluble CNTFR polypeptide.

26. The method according to claim 25, wherein the soluble CNTFR polypeptide comprises one or more mutations that reduce the binding affinity of the soluble CNTFR polypeptide for gp130, LIFR, or both.

27. The method according to claim 26, wherein the soluble CNTFR polypeptide comprises one or more mutations that reduce the binding affinity of the soluble CNTFR polypeptide for LIFR.

28. The method according to claim 27, wherein the one or more mutations that reduce binding affinity for LIFR is at amino acid position 177, 178, or both, relative to a CNTFR polypeptide having the amino acid sequence set forth in SEQ ID NO:8.

29. The method according to claim 26, wherein the soluble CNTFR polypeptide comprises one or more mutations that reduce the binding affinity of the soluble CNTFR polypeptide for gp130.

30. The method according to claim 29, wherein the one or more mutations that reduce binding affinity for gp130 is at amino acid position 268, 269, or both, relative to a CNTFR polypeptide having the amino acid sequence set forth in SEQ ID NO:8.

31. The method according to any one of claims 25 to 30, wherein the soluble CNTFR polypeptide comprises one or more mutations that increase the binding affinity of the soluble CNTFR polypeptide for CLCF1 relative to a CNTFR polypeptide having the amino acid sequence set forth in SEQ ID NO:8.

32. The method according to claim 31, wherein the one or more mutations that increase binding affinity for CLCF1 is at amino acid position 110, 174, 237, 287, or any combination thereof, relative to a CNTFR polypeptide having the amino acid sequence set forth in SEQ ID NO:8.

33. The method according to any one of claims 25 to 32, wherein the soluble CNTFR polypeptide specifically binds to CLCF1 and comprises an amino acid substitution selected from R110Q, T174P, Y177H, K178N, S237F, T268A, D269A, I287F, and any combination thereof, wherein numbering is as in SEQ ID NO:9, and wherein the soluble CNTFR polypeptide comprises 70% or greater, 75% or greater, 80% or greater, 85% or greater, 90% or greater, 95% or greater, 99% or greater, or 100% amino acid sequence identity to amino acids 23-342 of SEQ ID NO:9.

34. The method according to any one of claims 25 to 32, wherein the soluble CNTFR polypeptide specifically binds to CLCF1 and comprises the amino acid substitutions R110Q, T174P, Y177H, K178N, S237F, T268A, D269A, and I287F, wherein numbering is as in SEQ ID NO:9, and wherein the soluble CNTFR polypeptide comprises 70% or greater, 75% or greater, 80% or greater, 85% or greater, 90% or greater, 95% or greater, 99% or greater, or 100% amino acid sequence identity to amino acids 23-342 of SEQ ID NO:9.

35. The method according to any one of claims 25 to 32, wherein the soluble CNTFR polypeptide is fused to an Fc domain, specifically binds to CLCF1, and comprises an amino acid substitution selected from R110Q, T174P, Y177H, K178N, S237F, T268A, D269A, I287F, and any combination thereof, wherein numbering is as in SEQ ID NO:10, and wherein the soluble CNTFR polypeptide comprises 70% or greater, 75% or greater, 80% or greater, 85% or greater, 90% or greater, 95% or greater, 99% or greater, or 100% amino acid sequence identity to amino acids 23-578 of SEQ ID NO:10.

36. The method according to any one of claims 25 to 32, wherein the soluble CNTFR polypeptide is fused to an Fc domain, specifically binds to CLCF1, and comprises the amino acid substitutions R110Q, T174P, Y177H, K178N, S237F, T268A, D269A, and I287F, wherein numbering is as in SEQ ID NO:10, and wherein the soluble CNTFR polypeptide comprises 70% or greater, 75% or greater, 80% or greater, 85% or greater, 90% or greater, 95% or greater, 99% or greater, or 100% amino acid sequence identity to amino acids 23-578 of SEQ ID NO:10.

37. The method according to any one of claims 25 to 34, wherein the soluble CNTFR polypeptide comprises a solubility-conferring mutation in the domain that anchors wild-type CNTFR to a cell membrane.

38. The method according to claim 37, wherein the soluble CNTFR polypeptide comprises a truncation in the domain that anchors wild-type CNTFR to a cell membrane.

39. The method according to claim 37, wherein the soluble CNTFR polypeptide lacks the domain that anchors wild-type CNTFR to a cell membrane.

40. The method according to any one of claims 1 to 39, wherein the agent is a polypeptide fused to a heterologous polypeptide.

41. The method according to claim 40, wherein the heterologous polypeptide is an Fc domain, an albumin, a transferrin, XTEN, a homo-amino acid polymer, a proline-alanine-serine polymer, an elastin-like peptide, or any combination thereof.

42. The method according to claim 41, wherein the heterologous polypeptide is an Fc domain.

43. The method according to claim 42, wherein the Fc domain is a human Fc domain.

44. The method according to any one of claims 1 to 43, wherein the agent is conjugated to a moiety.

45. The method according to claim 44, wherein the moiety is polyethylene glycol (PEG), an anti-cancer drug, a detectable label, or any combination thereof.

46. A kit, comprising:
an agent that inhibits cardiotrophin-like cytokine factor 1 (CLCF1)-ciliary neurotrophic factor receptor (CNTFR) signaling; and
instructions for administering the agent to an individual identified as having a KRAS mutant cancer.

47. The kit of claim 46, wherein the agent is as defined in any one of claims 16 to 45.

48. The kit of claim 47, wherein the agent is a soluble CNTFR polypeptide as defined in any one of claims 25 to 45.

49. The kit of any one of claims 46 to 48, wherein the instructions comprise instructions for administering the agent to an individual identified as having a KRAS mutant lung cancer.

50. The kit of claim 49, wherein the instructions comprise instructions for administering the agent to an individual identified as having a KRAS mutant non-small cell lung cancer (NSCLC).

51. The kit of claim 50, wherein the instructions comprise instructions for administering the agent to an individual identified as having a KRAS mutant lung adenocarcinoma (LUAD).

52. The kit of any one of claims 46 to 51, wherein the instructions comprise instructions for administering the agent to an individual identified as having a KRAS mutant cancer comprising an amino acid substitution at position 12 of human KRAS, and wherein numbering is as in SEQ ID NO:1.

53. The kit of claim 52, wherein the instructions comprise instructions for administering the agent to an individual identified as having a KRAS mutant cancer comprising an amino acid substitution selected from the group consisting of: G12A, G12C, G12D, G12S , and G12V.
